(19) United States
(12) Patent Application Publication
(10) Pub. No.: US 2023/0399313 A1
(43) Pub. Date: Dec. 14, 2023

(54) BIOLOGICAL ACTIVITIES OF 5-(2-(4-(4-FLUORO-2-METHYL-1H-INDOL-5-YLOXY)-6-METHOXYQUINOLIN-7-YLOXY)ETHYL)-5-AZASPIRO[2.4]-HEPTAN-7-OL CRYSTALLINE, PHOSPHORIC ACID SALT AND ITS ENANTIOMERS

(71) Applicant: Advenchen Pharmaceuticals, LLC, Moorpark, CA (US)

(72) Inventors: Guoqing Paul Chen, Westlake Village, CA (US); Zhe Li, Los Angeles, CA (US); Yingyin Li, Hangzhou (CN); Lisha Qi, Nanjing (CN); Tingting Yu, Nanjing (CN)

(73) Assignee: Advenchen Pharmaceuticals, LLC, Moorpark, CA (US)

(21) Appl. No.: 18/206,506

(22) Filed: Jun. 6, 2023

Related U.S. Application Data
(60) Provisional application No. 63/351,205, filed on Jun. 10, 2022.

(51) Int. Cl. C07D 401/14; A61K 9/20; A61K 9/28; A61K 39/395; A61P 35/00; A61K 31/4709

(52) U.S. Cl. CPC C07D 401/14; A61K 9/2059; A61K 9/2054; A61K 9/2866; A61K 9/2018; A61K 9/2013; A61K 9/2009; A61K 39/3955; A61P 35/00; A61K 31/4709; C07B 2200/13

(57) ABSTRACT

Disclosed herein relates to the biological activities and preparation of 5-(2-(4-(4-fluoro-2-methyl-1H-indol-5-yloxy)-6-methoxyquinolin-7-yloxy)ethyl)-5-azaspiro[2.4]-heptan-7-ol (Compound A) stable crystalline form (Compound A-Xln); its tablet (Compound A-Xln tablet); its phosphoric acid salt (Compound A-P) and phosphoric acid salt stable crystalline form (Compound A-P-Xln); as well as its enantiomers (Compound R-A, Compound S-A). Furthermore, their anti-cancer activities, stabilities, chirality, dissolutions and treatment indications have also been investigated.

This application claims the benefit of U.S. provisional application: 63/351,205 filed on Jun. 10, 2022.

### FIELD OF THE INVENTION

The present disclosure relates to the biological activities and preparation of 5-(2-(4-(4-fluoro-2-methyl-1H-indol-5-yloxy)-6-methoxyquinolin-7-yloxy)ethyl)-5-azaspiro[2.4]-heptan-7-ol (Compound A) stable crystalline form (Compound A-Xln); its tablet (Compound A-Xln tablet); its phosphoric acid salt (Compound A-P) and phosphoric acid salt stable crystalline form (Compound A-P-Xln); as well as its enantiomers (Compound R-A, Compound S-A).

### BACKGROUND

Protein tyrosine kinases (PTKs) are a series of enzymes that catalyze the phosphorylation process of transferring phosphate groups from nucleoside triphosphates (often ATP) to protein amino acid residues. This phosphorylation process can activate the phosphorylated protein. Thus, PTKs play roles as “switches” to modulate many cellular functionalities by the means of controlling the signal cascades from extracellular through membrane to the inner part cytoplasm and even nucleus.

### SUMMARY

Several embodiments disclosed herein pertain to inhibitors of PTKs with improved stability. These inhibitors can be used to treat PTK mediated disorders and diseases. For instance, according to their locations on different extracellular domains, PTKs can be classified as epidermal growth factor receptor (EGFR), platelet-derived growth factor receptor (PDGFR), vascular endothelial growth factor receptor (VEGFR), fibroblast growth factor receptor (FGFR) and etc. Lots of studies have demonstrated the fact that normal cells usually show low activity or no activity of PTKs, whereas many tumor cells, especially gliomas and carcinomas, feature over expression of PTKs. Obviously, the unusual hyperactivity of PTKs is closely correlated with the tumor cell growth and angiogenesis process.

Interrupting or blocking the activity of PTKs could dramatically suppress the growth of tumor cells by the means of inhibiting cellular signal transductions. For this reason, targeted therapy to suppress the expression of PTKs via PTK inhibitors has become a widely accepted therapy.

The novel PTK inhibitor 5-(2-(4-(4-fluoro-2-methyl-1H-indol-5-yloxy)-6-methoxyquinolin-7-yloxy)ethyl)-5-azaspiro-[2.4]-heptan-7-ol (Compound A) features the potential to inhibit the activity of many protein tyrosine kinases (PTKs), including but not limit to VEGFr, EGFr, c-kit, PDGF, FGF, SRC, Aurora B and etc. The structure and synthetic route of 5-(2-(4-(4-fluoro-2-methyl-1H-indol-5-yloxy)-6-methoxyquinolin-7-yloxy)ethyl)-5-azaspiro-[2.4]-heptan-7-ol (Compound A) has already been disclosed in patent WO2010021918.

Several embodiments pertain to the stable crystal form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is made by a method comprising subjecting an amorphous form of 5-(2-(4-(4-fluoro-2-methyl-1H-indol-5-yloxy)-6-methoxyquinolin-7-yloxy)ethyl)-5-azaspiro[2.4]-heptan-7-ol via recrystallization.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is made by a method comprising two recrystallization steps.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is made by a method comprising a recrystallization process performed using a high boiling point solvent or a mixture of solvents that together have a high boiling point.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is made by a method comprising a recrystallization process performed using a low boiling point solvent or a mixture of solvents that together have a low boiling point.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is made by a method comprising a first recrystallization process and a second recrystallization process.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is made by a method comprising: a first recrystallization process with a high boiling point solvent or mixture of solvents that together have a high boiling point; and a second recrystallization process with a low boiling point solvent or mixture of solvents that together have a low boiling point. In several embodiments, the high boiling point solvent is DMF and the low boiling point solvent is EtOH.

Several embodiments pertain to a crystalline form of 5-(2-(4-(4-fluoro-2-methyl-1H-indol-5-yloxy)-6-methoxyquinolin-7-yloxy)ethyl)-5-azaspiro[2.4]-heptan-7-ol exhibiting at least one of the following properties: a DSC Melting Range (Endo): 240-260° C. with Peak Temp Range: 244-254° C.; more specifically, a DSC Melting Range (Endo): 247-253° C. with Peak Temp=249° C., the pattern is shown in FIG. 2 or a TGA thermogram demonstrating as an unsolvated material with weight loss above 250° C., the pattern is shown in FIG. 3.

Several embodiments pertain to a crystalline form of 5-(2-(4-(4-fluoro-2-methyl-1H-indol-5-yloxy)-6-methoxyquinolin-7-yloxy)ethyl)-5-azaspiro[2.4]-heptan-7-ol featuring an XRPD pattern as shown in FIG. 4.

Several embodiments pertain to a crystalline form of 5-(2-(4-(4-fluoro-2-methyl-1H-indol-5-yloxy)-6-methoxyquinolin-7-yloxy)ethyl)-5-azaspiro[2.4]-heptan-7-ol featuring an XRPD result comprising 14 characteristic peaks with intensity % greater than 10% expressed in d values and angles as follows:

| NO. | Angle | d value | Intensity | Intensity % |
|---|---|---|---|---|
| 1 | 8.899 | 9.9286 | 74540 | 12.04 |
| 2 | 9.927 | 8.9032 | 235708 | 38.08 |
| 5 | 14.938 | 5.9257 | 159717 | 25.80 |
| 6 | 15.374 | 5.7587 | 104484 | 16.88 |
| 7 | 16.104 | 5.4991 | 93019 | 15.03 |
| 8 | 16.558 | 5.3495 | 619004 | 100.00 |
| 11 | 18.492 | 4.7942 | 87559 | 14.15 |
| 13 | 19.912 | 4.4553 | 118882 | 19.21 |
| 14 | 20.445 | 4.3403 | 180671 | 29.19 |
| 15 | 21.511 | 4.1275 | 348507 | 56.30 |
| 19 | 23.859 | 3.7265 | 69270 | 11.19 |
| 20 | 24.314 | 3.6577 | 67756 | 10.95 |
| 21 | 24.590 | 3.6172 | 93287 | 15.07 |
| 23 | 25.952 | 3.4305 | 86263 | 13.94 |

Several embodiments pertain to a crystalline form of 5-(2-(4-(4-fluoro-2-methyl-1H-indol-5-yloxy)-6-methoxyquinolin-7-yloxy)ethyl)-5-azaspiro[2.4]-heptan-7-ol featuring a XRPD pattern as shown FIG. 11.

Several embodiments pertain to a crystalline form of 5-(2-(4-(4-fluoro-2-methyl-1H-indol-5-yloxy)-6-methoxyquinolin-7-yloxy)ethyl)-5-azaspiro[2.4]-heptan-7-ol featuring an XRPD result comprising 43 characteristic peaks with all intensity % expressed in d values and angles as follows:

| NO. | Angle | d value | Intensity | Intensity % |
|---|---|---|---|---|
| 1 | 8.899 | 9.9286 | 74540 | 12.04 |
| 2 | 9.927 | 8.9032 | 235708 | 38.08 |
| 3 | 11.586 | 7.6312 | 14908 | 2.41 |
| 4 | 13.145 | 6.7299 | 3511 | 0.57 |
| 5 | 14.938 | 5.9257 | 159717 | 25.80 |
| 6 | 15.374 | 5.7587 | 104484 | 16.88 |
| 7 | 16.104 | 5.4991 | 93019 | 15.03 |
| 8 | 16.558 | 5.3495 | 619004 | 100.00 |
| 9 | 17.327 | 5.1137 | 22260 | 3.60 |
| 10 | 17.820 | 4.9734 | 20322 | 3.28 |
| 11 | 18.492 | 4.7942 | 87559 | 14.15 |
| 12 | 18.809 | 4.7140 | 21872 | 3.53 |
| 13 | 19.912 | 4.4553 | 118882 | 19.21 |
| 14 | 20.445 | 4.3403 | 180671 | 29.19 |
| 15 | 21.511 | 4.1275 | 348507 | 56.30 |
| 16 | 22.004 | 4.0362 | 14250 | 2.30 |
| 17 | 22.499 | 3.9485 | 3195 | 0.52 |
| 18 | 23.188 | 3.8327 | 51679 | 8.35 |
| 19 | 23.859 | 3.7265 | 69270 | 11.19 |
| 20 | 24.314 | 3.6577 | 67756 | 10.95 |
| 21 | 24.590 | 3.6172 | 93287 | 15.07 |
| 22 | 25.171 | 3.5350 | 1707 | 0.28 |
| 23 | 25.952 | 3.4305 | 86263 | 13.94 |
| 24 | 26.464 | 3.3652 | 53165 | 8.59 |
| 25 | 26.841 | 3.3188 | 20232 | 3.27 |
| 26 | 27.217 | 3.2738 | 6582 | 1.06 |
| 27 | 27.648 | 3.2237 | 46760 | 7.55 |
| 28 | 28.457 | 3.1339 | 54339 | 8.78 |
| 29 | 29.310 | 3.0446 | 1984 | 0.32 |
| 30 | 29.856 | 2.9901 | 4264 | 0.69 |
| 31 | 30.212 | 2.9557 | 18700 | 3.02 |
| 32 | 31.202 | 2.8642 | 26858 | 4.34 |
| 33 | 31.632 | 2.8262 | 24054 | 3.89 |
| 34 | 32.837 | 2.7252 | 55773 | 9.01 |
| 35 | 33.430 | 2.6782 | 39092 | 6.32 |
| 36 | 35.185 | 2.5485 | 6426 | 1.04 |
| 37 | 36.092 | 2.4865 | 16774 | 2.71 |
| 38 | 36.865 | 2.4361 | 20105 | 3.25 |
| 39 | 38.167 | 2.3560 | 16104 | 2.60 |
| 40 | 39.409 | 2.2845 | 5045 | 0.82 |
| 41 | 40.888 | 2.2053 | 5238 | 0.85 |
| 42 | 41.720 | 2.1632 | 8720 | 1.41 |
| 43 | 42.886 | 2.1070 | 9964 | 1.61 |

In several embodiments, the crystal form of 5-(2-(4-(4-fluoro-2-methyl-1H-indol-5-yloxy)-6-methoxyquinolin-7-yloxy)ethyl)-5-azaspiro[2.4]-heptan-7-ol has improved better stability and/or solubility than its amorphous form.

In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is a pharmaceutical acceptable salt. In several embodiments, the stable crystal form of 5-(2-(4-(4-fluoro-2-methyl-1H-indol-5-yloxy)-6-methoxyquinolin-7-yloxy)ethyl)-5-azaspiro[2.4]-heptan-7-ol is the phosphoric acid salt.

In several embodiments, the phosphoric acid salt is prepared by a method including a step for neutralization of a solution of phosphoric acid in solvent or a mixture of solvents and the process of recrystallization.

In several embodiments, the solvent in which neutralization and/or recrystallization performed is EtOH.

In several embodiments, the phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol is characterized by one or more of the following properties: a DSC Melting Range (Endo): 221-235° C. with Peak Temp=229° C., pattern was shown in FIG. 9; a TGA demonstrating as a slight weight loss at about 30-60° C. and significantly weight loss above 210° C., the pattern was shown in FIG. 10.

In several embodiments, the phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol comprises a XRPD pattern which was shown in FIG. 11. XRPD result compromising all characteristic peaks with all intensity % expressed in d values and angles as follows:

| NO. | Angle | d value | Intensity | Intensity % |
|---|---|---|---|---|
| 1 | 5.268 | 16.760 | 83.1 | 26.6 |
| 2 | 7.139 | 12.372 | 251.0 | 80.6 |
| 3 | 9.805 | 9.014 | 94.9 | 30.4 |
| 4 | 10.455 | 8.455 | 119.0 | 38.2 |
| 5 | 11.799 | 7.494 | 98.1 | 31.4 |
| 6 | 12.417 | 7.123 | 181.0 | 58.1 |
| 7 | 12.669 | 6.982 | 225.0 | 72.0 |
| 8 | 13.672 | 6.472 | 280.0 | 89.8 |
| 9 | 14.307 | 6.186 | 136.0 | 43.5 |
| 10 | 15.546 | 5.695 | 137.0 | 44.0 |
| 11 | 16.064 | 5.513 | 181.0 | 58.1 |
| 12 | 16.719 | 5.298 | 312.0 | 100.0 |
| 13 | 17.495 | 5.065 | 135.0 | 43.4 |
| 14 | 18.035 | 4.915 | 136.0 | 43.7 |
| 15 | 18.802 | 4.716 | 153.0 | 48.9 |
| 16 | 19.530 | 4.542 | 285.0 | 91.3 |
| 17 | 21.095 | 4.208 | 200.0 | 64.1 |
| 18 | 22.535 | 3.942 | 138.0 | 44.2 |
| 19 | 23.426 | 3.794 | 294.0 | 94.3 |
| 20 | 25.916 | 3.435 | 114.0 | 36.6 |
| 21 | 26.577 | 3.351 | 144.0 | 46.3 |

Several embodiments pertain to a compound having a structure represented by 5-(2-(4-(4-fluoro-2-methyl-1H-indol-5-yloxy)-6-methoxyquinolin-7-yloxy)ethyl)-5-azaspiro[2.4]-heptan-7-ol, its stable crystalline form, its salt form, and/or its stable crystalline salt form, for use in inhibiting protein tyrosine kinases (PTKs). In several embodiments, the PTKs are selected but not limited to FGFR1(h), FGFR2(h), FGFR3(h), Flt1(h) (VEGFr1), Flt4(h) (VEGFr3), KDR(h) (VEGFr2), Aurora-B PDGFRα(h), PDGFRα(h) and PDGFRβ(h). In several embodiments, the salt form is the phosphoric acid salt form.

Several embodiments pertain to a compound having a structure represented by 5-(2-(4-(4-fluoro-2-methyl-1H-indol-5-yloxy)-6-methoxyquinolin-7-yloxy)ethyl)-5-azaspiro[2.4]-heptan-7-ol, its stable crystalline form, its salt form, and/or its stable crystalline salt form, wherein the compound inhibits common cancer cell lines, including but not limited to PANC-1, NCI-H157, MDA-MB-231, Hela, PC-3, BEL7404, MKN45, Ishikawa, Saos-2, SKOV3, SW579 and HCT116. In several embodiments, the salt form is the phosphoric acid salt form.

Several embodiments pertain to a pharmaceutical composition that comprises as an active ingredient that is a compound of as described above or elsewhere herein and a pharmaceutically acceptable carrier and/or pharmaceutically acceptable excipient.

Several embodiments pertain to a pharmaceutical composition that comprises as an active ingredient that is a compound of as described above or elsewhere herein and a pharmaceutically acceptable carrier and/or pharmaceutically acceptable excipient to form a tablet.

Several embodiments pertain to a pharmaceutical composition that comprises as an active ingredient selected from a stable crystalline free base form of 5-(2-(4-(4-fluoro-2-methyl-1H-indol-5-yloxy)-6-methoxyquinolin-7-yloxy)ethyl)-5-azaspiro[2.4]-heptan-7-ol or a stable crystalline form of phosphoric acid salt and a pharmaceutically acceptable carrier.

Several embodiments pertain to a method of treating a neoplastic disease, comprising administering a compound as defined above or elsewhere herein or pharmaceutical composition comprising such a compound and pharmaceutically acceptable excipients to a subject in need thereof. In several embodiments, the method comprises administration of an additional agent, the additional agent is a chemotherapy compound and/or immunotherapy agents. In several embodiments, the neoplastic disease is solid tumors, selected from lung, renal, colorectal, gastric, melanoma, head/neck, thyroid, pancreatic, liver, prostate, bladder, brain, sarcoma, breast, ovarian, cervical and endometrial cancers; and blood cancers, selected from ALL, CLL, AML, CML and Multiple Myeloma, especially in human clinical studies to treat small cell lung cancer (SCLC), non-small cell lung cancer (NSCLC) and ovarian cancer. Several embodiments pertain to a method of treating, wherein the combination chemotherapy agents are selected from platinum-based or taxane-based or topoisomerase 1 inhibitor or alkaloid or alkylating agents. In several embodiments, the chemotherapy agents are selected from cisplatin, carboplatin, paclitaxel or cisplatin/paclitaxel or carboplatin/paclitaxel or carboplatin/etoposide or topotecan, irinotecan or lomustine. In several embodiments, the immunotherapy agents are selected from PD-1 or PD-L1 antibodies including but not limited to nivolumab, pembrolizumab, ipilimumab, blinatumomab, elotuzumab, daratumumab, cemiplimab, avelumab, durvalumab, atezolizumab, toripalimab, sintilimab, camrelizumab, tislelizumab, AK104, Penpulimab, KN035, CS1001, talimogene laherparepvec.

### BRIEF DESCRIPTION OF THE DRAWINGS

FIG. 1. DSC graph of an amorphous (Compound A-Amp) form of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 2. DSC graph of a crystalline form of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 3. TGA graph of a crystalline form of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 4. XRPD graph of a crystalline form of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 5. ¹H-HMR Spectra of a crystalline form of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 6. Stability test of a amorphous API and a crystalline API of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 7. Stability test of an amorphous tablet and a crystalline tablet of 5-(2-(4-(4-fluoro-2-methyl-1H-indol-5-yloxy)-6-methoxyquinolin-7-yloxy)ethyl)-5-azaspiro[2.4]-heptan-7-ol.

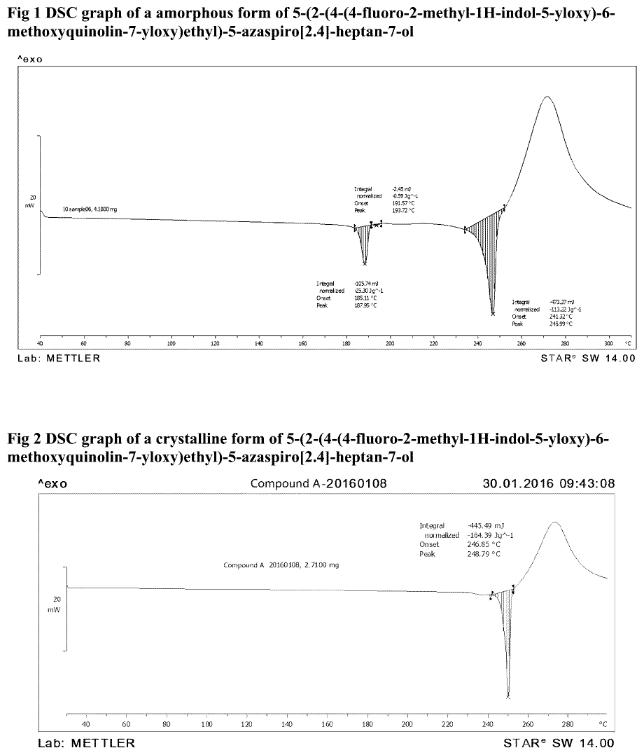

FIG. 1. Dissolution curves of a amorphous tablet and a crystalline tablet of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 9. DSC graph of a crystalline form of phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 10. TGA graph of a crystalline form of phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 11. XRPD graph of a crystalline form of phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 12. ¹H-HMR Spectra of a crystalline form of phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol.

FIG. 13. Proliferation inhibition of breast cancer cell line MCF7 by Compound A-Xln, anti-breast cancer agent Anatrozole and Letrozole, combination of Compound A-Xln and Anatrozole, and combination of Compound A-Xln and Letrozole.

FIG. 14. Proliferation inhibition of breast cancer cell line BT474 by Compound A-Xln, anti-breast cancer agent Anatrozole and Letrozole, combination of Compound A-Xln and Anatrozole, and combination of Compound A-Xln and Letrozole.

FIG. 15. Proliferation inhibition of colon cancer cell line HT29 by Compound A-Xln, anti-colon cancer agent 5FU/O, and combination of Compound A-Xln and 5FU/O.

FIG. 16. Proliferation inhibition of colon cancer cell line HCT116 by Compound A-Xln, anti-colon cancer agent 5FU/O, and combination of Compound A-Xln and 5FU/O.

FIG. 17. Proliferation inhibition of colon cancer cell line colo205 by Compound A-Xln, anti-colon cancer agent 5FU/O, and combination of Compound A-Xln and 5FU/O.

FIG. 18. Comparison of mean tumor volume changes following administration of Compound A-Xln, an anti-PD1 antibody agent, and combinational therapy of Compound A-Xln and an anti-PD1 antibody agent against murine colorectal CT26 cell line xenograft model.

FIG. 19. Relative tumor volume changes following treatment of Compound A-Xln against small cell lung cancer NCI-H1436 cell line xenograft model

FIG. 20. Relative tumor volume changes following treatment of Compound A-Xln against non small cell lung cancer (NSCLC) 95D cell line xenograft model

FIG. 21. Relative tumor volume changes following treatment of Compound A-Xln against ovarian cancer SKOV3 cell line xenograft model

FIG. 22. Relative tumor volume changes following treatment of Compound A-Xln against renal cancer 786-O cell line xenograft model

FIG. 23. Relative tumor volume changes following treatment of Compound A-Xln against liver cancer Bel-7402 cell line xenograft model

FIG. 24. Relative tumor volume changes following treatment of Compound A-Xln against glioblastoma U87 cell line xenograft model

FIG. 25. Kaplan-Meier Progression Free Survival (PFS) Analysis Curve For Group C (≥2nd prior line, n=30) & B (=1st prior line, n=30) on SCLC patients

FIG. 26. Kaplan-Meier Overall Survival (OS) Analysis Curve For Group C (≥2nd prior line, n=30) & B (=1st prior line, n=30) on SCLC patients

### DETAILED DESCRIPTION

Several embodiments relate to compound 5-(2-(4-(4-fluoro-2-methyl-1H-indol-5-yloxy)-6-methoxyquinolin-7-yloxy)ethyl)-5-azaspiro[2.4]-heptan-7-ol (Compound A), its stable crystalline form (Compound A-Xln) and amorphous from (Compound A-Amp), its stabilized crystalline form (Compound A-P-Xln) which crystlized from a phosphoric acid salt (Compound A-P); and enantiomers (two chiral isomers) of any of the foregoing phosphoric acid salt or crystallines (e.g., Compound R-A, Compound S-A, Compound R-A-Xln, Compound S-A-Xln, Compound R-A-P-Xln, Compound S-A-P-Xln). Compound A is represented by the following structure:

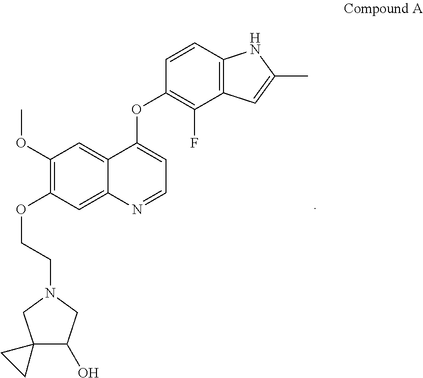 [SMILES: COc1cc2c(Oc3ccc4[nH]c(C)cc4c3F)ccnc2cc1OCCN1CC(O)C2(CC2)C1]

The following description provides context and examples but should not be interpreted to limit the scope of the inventions covered by the claims that follow in this specification or in any other application that claims priority to this specification. No single component (including a method step) or collection of components (e.g., multiple steps) is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

The section headings used herein are for organizational purposes only and are not to be construed as limiting the subject matter described. Features disclosed under one heading (such as a composition) can be used in combination with features disclosed under a different heading (a method of treating). Unless defined otherwise, all technical and scientific terms used herein have the same meaning as commonly understood by one of ordinary skill in the art. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

When referring to various features, the terms “or ranges including and/or spanning the aforementioned values” may be used. These terms (and variations thereof) are meant to include any range that includes or spans any of the aforementioned values. For example, with regard to a temperature, the temperature may be expressed as “equal to or at least about: 50° C., 60° C., 70° C., or ranges including and/or spanning the aforementioned values.” This language includes not only the particular temperature provided and the range exceeding that temperature (e.g., equal to or at least about 40° C., equal to or at least about 50° C., equal to or at least about 60° C., and equal to or at least about 70° C.) but also the temperature ranges spanning those values (e.g., from 40° C. to 50° C., from 40° C. to 60° C., from 40° C. to 70° C., from 50° C. to 60° C., from 50° C. to 70° C., or 60° C. to 70° C.). Similarly, with regard to a temperature, the temperature may be expressed as “equal to or less than about: 40° C., 50° C., 70° C., or ranges including and/or spanning the aforementioned values.” This language includes not only the particular temperature provided and the range below that value (e.g., equal to or less than about 40° C., equal to or less than about 50° C., equal to or less than about and equal to or less than about 70° C.) but also temperature ranges spanning those values (e.g., from 40° C. to 50° C., from 40° C. to 60° C., from 40° C. to 70° C., from 50° C. to 60° C., from 50° C. to 70° C., or 60° C. to 70° C.).

The term “pharmaceutically acceptable carrier” or “pharmaceutically acceptable excipient” includes any solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects.

The term “therapeutically effective amount,” as used herein, refers to an amount of the therapeutic that imparts a modulating effect, which, for example, can be a beneficial effect, to a subject afflicted with a disorder, disease or illness, including improvement in the condition of the subject (e.g., modulating one or more symptoms), delay or reduction in the progression of the condition, prevention or delay of the onset of the disorder, and/or change in clinical parameters, disease or illness, etc. For example, in some embodiments, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Actual dosage levels of active ingredients and agents in an active composition of the disclosed subject matter can be varied so as to administer an amount of the active agent(s) that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. Determination and adjustment of an effective dose, as well as evaluation of when and how to make such adjustments, are contemplated herein. The term “a therapeutically effective amount” can mean an amount of therapeutic sufficient to prevent the spread of or reverse cancer.

### Compounds

Several embodiments relate to therapeutic compounds (e.g., therapeutics). Several embodiments pertain to 5-(2-(4-(4-fluoro-2-methyl-1H-indol-5-yloxy)-6-methoxyquinolin-7-yloxy)ethyl)-5-azaspiro[2.4]-heptan-7-ol (Compound A), its crystalline form (Compound A-Xln), its stable crystalline form (Compound A-P-Xln) of phosphoric acid salt (Compound A-P) and enantiomer of any of the foregoing (e.g., Compound R-A, Compound S-A, Compound R-A-Xln, Compound S-A-Xln, Compound R-A-P-Xln, Compound S-A-P-Xln), each of which is a therapeutic compound. In several embodiments, an S in the Compound A name indicates the S configuration and an R in the Compound A name indicates the R configuration. The two enantiomers of Compound A, Compound R-A and Compound S-A are further represented by the following structures, respectively:

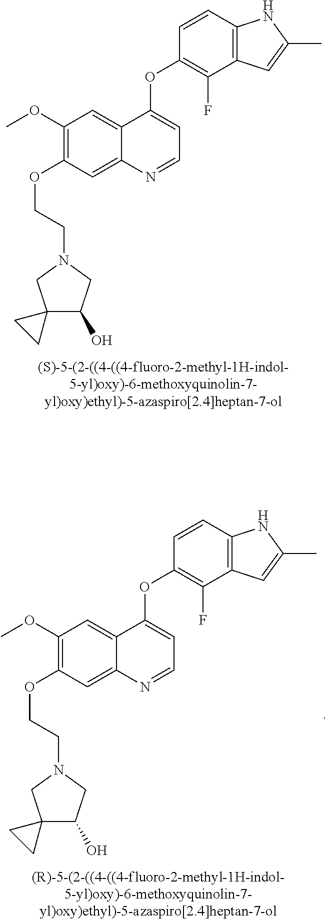

In several embodiments, a crystalline form of Compound A is provided. In several embodiments, the crystal form of Compound A is the free base form. In several embodiments, the crystal form is expressed as Compound A-Xln. In several embodiments, the crystal form is characterized by one or more peaks in an XRPD pattern. In several embodiments, the crystal form is characterized by XRPD peaks at angles selected from one or more of 8.899, 9.927, 11.586, 13.145, 14.938, 15.374, 16.104, 16.558, 17.327, 17.820, 18.492, 18.809, 19.912, 20.445, 21.511, 22.004, 22.499, 23.188, 23.859, 24.314, 24.590, 25.952, 26.464, 26.841, 27.217, 27.648, 28.457, 29.310, and 29.856, or combinations of any of the foregoing. In several embodiments, the crystal form is characterized by XRPD peaks with intensities over 10% at angles selected from one or more of 8.899, 9.927, 14.938, 16.104, 16.558, 18.492, 19.912, 20.445, 21.511, 23.859, 24.314, 24.590, and 25.952, or combinations of any of the foregoing. In several embodiments, the crystal form is characterized by XRPD peaks with intensities over 20% at angles selected from one or more of 9.927, 14.938, 16.558, 20.445, and 21.511, or combinations of any of the foregoing.

In several embodiments, the DSC melting range of Compound A-Xln, is characterized by a melting range equal to or less than about 245° C., 250° C., 255° C., or ranges including and/or spanning the aforementioned values. In several embodiments, the DSC melting range of Compound A-Xln, is characterized by a melting point of 249° C.

In several embodiments, the TGA peak weight loss range of Compound A-Xln, is equal to or less than about 245° C., 250° C., 255° C., or ranges including and/or spanning the aforementioned values. In several embodiments, the TGA peak weight loss Compound A-Xln, occurs at a temperature of 249° C.

In several embodiments, a crystalline form of a salt of Compound A is provided. In several embodiments, the crystal form is expressed as Compound A-P-Xln. In several embodiments, the crystal form is characterized by one or more peaks in an XRPD pattern. In several embodiments, the crystal form is characterized by XRPD peaks at angles selected from one or more of 5.268, 7.139, 9.805, 10.455, 11.799, 12.417, 12.669, 13.672, 14.307, 16.064, 16.719, 17.495, 18.035, 18.802, 19.530, 21.095, 22.535, 23.426, 25.916, and 26.577, or combinations of any of the foregoing. In several embodiments, the crystal form is characterized by XRPD peaks with intensities over 50% at angles selected from one or more of 7.139, 12.417, 12.669, 13.672, 16.064, 16.719, 19.530, 21.095, and 23.426, or combinations of any of the foregoing. In several embodiments, the crystal form is characterized by XRPD peaks with intensities over 80% at angles selected from one or more of 7.139, 13.672, 19.530, and 23.426, or combinations of any of the foregoing.

In several embodiments, the DSC melting range of Compound A-P-Xln, is characterized by a melting range equal to or less than about 220° C., 225° C., 230° C., 235° C., 240° C., 245° C., or ranges including and/or spanning the aforementioned values. In several embodiments, the DSC melting range of Compound A-P-Xln is characterized by a melting point of 229° C.

In several embodiments, the TGA peak weight loss range of Compound A-P-Xln is equal to or less than about: 205° C., 210° C., 215° C., 220° C., 225° C., 230° C., or ranges including and/or spanning the aforementioned values. In several embodiments, the TGA peak weight loss of Compound A-P-Xln, Compound occurs at a temperature of 210° C.

### Methods of Making Crystalline Forms

Several embodiments pertain to methods for the preparation of a stable crystal form of 5-(2-(4-(4-fluoro-2-methyl-1H-indol-5-yloxy)-6-methoxyquinolin-7-yloxy)ethyl)-5-azaspiro-[2.4]-heptan-7-ol (Compound A), such as Compound A-Xln, Compound S-A-Xln, Compound R-A-Xln, Compound A-P-Xln, Compound S-A-P-Xln, or Compound R-A-P-Xln, from amorphous form of Compound A (Compound A-Amp). In several embodiments, the method of making a stable crystalline form of Compound A comprises one or more crystallization and/or recrystallization steps.

In several embodiments, the preparation of a stable crystalline form of Compound A (e.g., Compound A-Xln, Compound S-A-Xln, Compound R-A-Xln, Compound A-P-Xln, Compound S-A-P-Xln, or Compound R-A-P-Xln) comprises at least one recrystallization step. In several embodiments, the method of making Compound A-Xln comprises more than one recrystallization step (e.g., 2 steps, 3 steps, 4 steps, etc.). In several embodiments, the preparation of a stable crystal form of Compound A (e.g., Compound A-Xln, Compound S-A-Xln, Compound R-A-Xln, Compound A-P-Xln, Compound S-A-P-Xln, or Compound R-A-P-Xln) comprises at least two recrystallization processes (e.g., two recrystallization steps). In other embodiments, the preparation only comprises a single recrystallization step.

In several embodiments, the preparation of a stable crystalline form of Compound A as disclosed herein (e.g., Compound A-Xln, Compound A-P-Xln) may include a recrystallization process in a high boiling point solvent. In several embodiments, a high boiling point solvent is a solvent (or mixture of solvents) with a boiling point that is equal to or at least about: 100° C., 110° C., 120° C., 130° C., 140° C., 150° C., 160° C., 170° C., 180° C., 190° C., 200° C., 250° C., or ranges including and/or spanning the aforementioned values. In several embodiments, after this recrystallization step, a stable form of one or more of Compound A-Xln is provided. In several embodiments, to provide a stable form of Compound A-P-Xln, Compound S-A-P-Xln, or Compound R-A-P-Xln, an additional recrystallization step is performed.

In several embodiments, the preparation of a stable crystalline form of Compound A as disclosed herein (e.g., Compound A-Xln, Compound S-A-Xln, Compound R-A-Xln, Compound A-P-Xln, Compound S-A-P-Xln, or Compound R-A-P-Xln) may include a recrystallization process in a low boiling point solvent (or a mixture of selected solvents). In several embodiments, a low boiling point solvent is a solvent (or mixture of solvents) with a boiling point that is equal to or less than about: 30° C., 40° C., 50° C., 60° C., 70° C., 80° C., 90° C., <100° C., or ranges including and/or spanning the aforementioned values.

In several embodiments, where at least two crystallization processes are used to prepare the stable crystal form of Compound A, one process may include a recrystallization process in a high boiling point solvent (e.g., a solvent with a high boiling point or mixture of solvents that together have a high boiling point) and another may include a recrystallization process in a low boiling point solvent (solvent having a low boiling point or mixture of solvents that together have a low boiling point). In several embodiments, where multiple recrystallization processes are used, an initial recrystallization process may be performed using a high boiling point solvent (or a mixture of selected solvents, including a high boiling solvent and another solvent that may or may not be high boiling). In several embodiments, where two or more recrystallization processes are used, the second recrystallization process may be performed using a low boiling point solvent (or a mixture of selected solvents, including a low boiling solvent and another solvent that may or may not be low boiling).

Alternatively, a recrystallization in a low boiling point solvent may precede a recrystallization in a high boiling point solvent.

As disclosed elsewhere herein, where mixtures of solvents are used, the mix of constituents may still be considered a “high boiling point solvent” when the mixture itself has a high boiling point. Likewise, where mixtures of solvents are used, the mix of constituents may still be considered a “low boiling point solvent” when the mixture itself has a low boiling point.

In several embodiments, for the preparation of a stable crystalline form (Compound A-Xln) of 5-(2-(4-(4-fluoro-2-methyl-1H-indol-5-yloxy)-6-methoxyquinolin-7-yloxy)ethyl)-5-azaspiro-[2.4]-heptan-7-ol (Compound A), the high boiling point solvent (e.g., for the initial recrystallization process) is selected from, but not limited to, DMF, DMA, NMP, a mixture of any of the foregoing selected solvents, or others. In several embodiments, for the preparation of a stable crystalline form (Compound A-Xln) of 5-(2-(4-(4-fluoro-2-methyl-1H-indol-5-yloxy)-6-methoxyquinolin-7-yloxy)ethyl)-5-azaspiro [2. 4]-heptan-7-ol (Compound A), the low boiling point solvent (e.g., for the second recrystallization process) is selected from, but not limited to, MeOH, EtOH, IPA, a mixture of any of the foregoing selected solvents, or others.

In several embodiments, for the preparation of a stable crystalline form of 5-(2-(4-(4-fluoro-2-methyl-1H-indol-5-yloxy)-6-methoxyquinolin-7-yloxy)ethyl)-5-azaspiro-[2.4]-heptan-7-ol (Compound A), the high boiling point solvent for the initial recrystallization process is DMF and the low boiling point solvent for the sequential recrystallization process (the second recrystallization process) is EtOH.

Different crystalline forms of a drug feature different physical and chemical properties (such as stability, solubility, dissolution rate, bioavailability and etc.) and thus leading to differences in the effectiveness, safety or quality of the drug. In several embodiments, one or more stability tests and/or dissolution tests may be performed. It has been found that the crystalline form of Compound A-Xln has better stability and dissolution rate comparing with the amorphous form of Compound A-Amp.

In several embodiments, as disclosed elsewhere herein, the disclosure relates to a method of preparing a salt form of 5-(2-(4-(4-fluoro-2-methyl-1H-indol-5-yloxy)-6-methoxy-quinolin-7-yloxy)ethyl)-5-azaspiro[2.4]-heptan-7-ol (Compound A), including a pharmaceutically acceptable salt of compounds of Compound A. The conception of “pharmaceutically acceptable salts” includes, but not limited to acid addition salts formed from inorganic acids such as hydrochloric acid, hydrobromic acid, sulfuric acid, nitric acid, phosphoric acid or the like; or acid addition salts formed from organic acids such as 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, oleic acid, oxalic acid, palmitic acid, pamoic acid, phosphoric acid, proprionic acid, pyroglutamic acid (−L), salicylic acid, sebacic acid, stearic acid, succinic acid, tartaric acid (+L), thiocyanic acid, toluenesulfonic acid (para-), undecylenic acid and the like.

Several embodiments relate to preparing a salt and/or a stable crystalline salt form of 5-(2-(4-(4-fluoro-2-methyl-1H-indol-5-yloxy)-6-methoxyquinolin-7-yloxy)ethyl)-5-azaspiro[2.4]-heptan-7-ol. Several embodiments relate to preparing a phosphoric acid salt (Compound A-P) and/or a stable crystalline phosphate salt form (Compound A-P-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A).

In several embodiments, for the preparation of a stable crystalline phosphate salt form (Compound A-P-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A), the method includes a neutralization process of a solution of Compound A and phosphoric acid in a solvent (or a mixture of solvents). In several embodiments, the recrystallization of Compound A-P is performed in a solvent (or a mixture of solvents).

In several embodiments, for the preparation of a stable crystalline phosphate salt form (Compound A-P-Xln) of 5-(2-(4-(4-fluoro-2-methyl-1H-indol-5-yloxy)-6-methoxyquinolin-7-yloxy)ethyl)-5-aza-spiro[2.4]-heptan-7-ol (Compound A), the solvent for the neutralization process and/or the recrystallization process includes a solvent selected from, but not limited, to MeOH, EtOH, IPA, a mixture of any of the foregoing, or other solvents.

In several embodiments, for the preparation of a stable crystalline phosphate salt form (Compound A-P-Xln) of 5-(2-(4-(4-fluoro-2-methyl-1H-indol-5-yloxy)-6-methoxyquino-lin-7-yloxy)ethyl)-5-azaspiro[2.4]-heptan-7-ol (Compound A), the solvent for the neutralization process and/or the recrystallization process is EtOH.

In several embodiments, as disclosed elsewhere herein, Compound A-Xln, Compound S-A-Xln, or Compound R-A-Xln may be prepared using a single recrystallization step (e.g., in a high boiling point solvent or mixture of solvents). In several embodiments, the Compound A is heated in the high boiling solvent. In several embodiments, a portion of the high boiling solvent is removed (including under vacuum). In several embodiments, the solution is allowed to rest (e.g., cool), at which time crystals form. In several embodiments, the crystals are collected.

In several embodiments, Compound A-P-Xln, Compound S-A-P-Xln, or Compound R-A-P-Xln may be prepared using subsequent recrystallization processes. In several embodiments, for example, Compound A-P-Xln, Compound S-A-P-Xln, or Compound R-A-P-Xln are prepared by introducing phosphoric acid (e.g., excess phosphoric acid) into the second recrystallization solvent (e.g., the low boiling point solvent). In several embodiments, the Compound A is heated in the solvent (e.g., the low boiling solvent). In several embodiments, a portion of the solvent is removed (including under vacuum). In several embodiments, the solution is allowed to rest (e.g., cool), at which time crystals form. In several embodiments, the crystals are collected. In several embodiments, where excess phosphoric acid is used, some of the phosphoric acid is neutralized.

### Pharmaceutical Compositions

In several embodiments, a pharmaceutical composition is provided. In several embodiments, the pharmaceutical composition comprises a stable crystalline form of Compound A or a stable crystalline form of a salt of Compound A as disclosed elsewhere herein and a pharmaceutically acceptable carrier. In several embodiments, the pharmaceutical composition comprises a stable crystalline form of Compound A or a stable crystalline form of a salt of Compound A as disclosed elsewhere herein and a pharmaceutically acceptable excipient.

In several embodiments, a method for preparing a pharmaceutical composition that comprises a stable crystalline form (Compound A-P-Xln) of 5-(2-(4-(4-fluoro-2-methyl-1H-indol-5-yloxy)-6-methoxyquinolin-7-yloxy)ethyl)-5-azaspiro[2.4]-heptan-7-ol, or a phosphoric acid salt (Compound A-P) or a stable crystalline phosphate salt form (Compound A-P-Xln) of 5-(2-(4-(4-fluoro-2-methyl-1H-indol-5-yloxy)-6-methoxyquinolin-7-yloxy)ethyl)-5-azaspiro[2.4]-heptan-7-ol, and a pharmaceutically acceptable carrier or excipient are provided. In several embodiments, an excipient or carrier (or both) are mixed with Compound A-Xln or Compound A-P-Xln.

### Methods of Treatment

In several embodiments, a method for treatment is provided. In several embodiments, the method includes acquiring a pharmaceutical composition that comprises a stable crystalline form (Compound A-P-Xln) of 5-(2-(4-(4-fluoro-2-methyl-1H-indol-5-yloxy)-6-methoxyquinolin-7-yloxy)ethyl)-5-azaspiro[2.4]-heptan-7-ol, or a phosphoric acid salt (Compound A-P) or a stable crystalline phosphate salt form (Compound A-P-Xln) of 5-(2-(4-(4-fluoro-2-methyl-1H-indol-5-yloxy)-6-methoxyquinolin-7-yloxy)ethyl)-5-azaspiro[2.4]-heptan-7-ol. In several embodiments, the method comprises administering Compound A-Xln, Compound A-P, or Compound A-P-Xln to a patient suffering from a disease. In several embodiments, the disease is a neoplastic disease.

In several embodiments, Compound A-Xln, Compound A-P, or Compound A-P-Xln include a single chiral isomer of one of Compound A-Xln, Compound A-P, or Compound A-P-Xln. In several embodiments, a method of preparing the chiral isomer is performed. In several embodiments, to separate enantiomers (two chiral isomers, Compound R-A, Compound S-A) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) from each other, a preparative HPLC with a chiral column. In several embodiments, the chiral isomers can then be recrystallized (and/or crystallized) as disclosed elsewhere herein.

Several embodiments relate to a stable crystalline form Compound A-Xln of 5-(2-(4-(4-fluoro-2-methyl-1H-indol-5-yloxy)-6-methoxyquinolin-7-yloxy)ethyl)-5-azaspiro[2.4]-heptan-7-ol, or a phosphoric acid salt or a stable crystalline phosphate salt form, or two chiral isomers (Compound R-A, Compound S-A) of 5-(2-(4-(4-fluoro-2-methyl-1H-indol-5-yloxy)-6-methoxyquinolin-7-yloxy)ethyl)-5-azaspiro[2.4]-heptan-7-ol, for use in the manufacture of amedicament for a method in treating a neoplastic disease. Several embodiments pertain to a method of treating a disease selected from the group consisting of lung cancer, renal cancer, colorectal cancer, gastric cancer, melanoma, head/neck cancer, thyroid cancer, pancreatic cancer, liver cancer, prostate cancer, bladder cancer, brain cancer, sarcoma, breast cancer, ovarian cancer, cervical cancer, endometrial cancer, especially small cell lung cancer (SCLC), non-small cell lung cancer (NSCLC) and ovarian cancer; tumors caused by any one or more of the foregoing, blood cancers, (selected from ALL, CLL, AML, CML and Multiple Myeloma), and combinations thereof. In several embodiments, the method of treating comprises administering a stable crystalline form Compound A-Xln of 5-(2-(4-(4-fluoro-2-methyl-1H-indol-5-yloxy)-6-methoxyquinolin-7-yloxy)ethyl)-5-azaspiro[2.4]-heptan-7-ol, or a phosphoric acid salt or a stable crystalline phosphate salt form, or two chiral isomers (Compound R-A, Compound S-A) of 5-(2-(4-(4-fluoro-2-methyl-1H-indol-5-yloxy)-6-methoxyquinolin-7-yloxy)ethyl)-5-azaspiro[2.4]-heptan-7-ol.

Several embodiments relate to a stable crystalline form or the salts or stable crystalline salt forms or two chiral isomers (Compound R-A, Compound S-A) of 5-(2-(4-(4-fluoro-2-methyl-1H-indol-5-yloxy)-6-methoxyquinolin-7-yloxy)ethyl)-5-azaspiro[2.4]-heptan-7-ol for a method in mono therapy or in combination with additional chemotherapy agents. In several embodiments, the additional chemotherapeutic agent is selected from platinum-based or taxane-based agents.

In several embodiments, the method of treating includes selecting a patient suffering from a disease selected comprising solid tumors. In several embodiments, the method of treating includes selecting a patient suffering from a disease selected from the group consisting of lung cancer, renal cancer, colorectal cancer, gastric cancer, melanoma, head/neck cancer, thyroid cancer, pancreatic cancer, liver cancer, prostate cancer, bladder cancer, brain cancer, sarcoma, breast cancer, ovarian cancer, cervical cancer, endometrial cancer, especially small cell lung cancer (SCLC), non-small cell lung cancer (NSCLC) and ovarian cancer; tumors caused by any one or more of the foregoing, blood cancers, (selected from ALL, CLL, AML, CML and Multiple Myeloma), and combinations thereof. In several embodiments, the method comprises administering a therapeutic amount of a stable crystalline form of 5-(2-(4-(4-fluoro-2-methyl-1H-indol-5-yloxy)-6-methoxyquinolin-7-yloxy)ethyl)-5-azaspiro[2.4]-heptan-7-ol, or a phosphoric acid salt or a stable crystalline phosphate salt form, or two chiral isomers (Compound R-A, Compound S-A) of 5-(2-(4-(4-fluoro-2-methyl-1H-indol-5-yloxy)-6-methoxyquinolin-7-yloxy)ethyl)-5-azaspiro [2.4]-heptan-7-ol to the patient.

In several embodiments, an effective amount of Compound A-Xln, Compound A-P, and/or Compound A-P-Xln is administered to a patient in need of treatment (e.g., a subject suffering from a disease as disclosed herein). In several embodiments, an effective amount of Compound A-Xln, Compound A-P, and/or Compound A-P-Xln is administered to a patient in need of treatment (e.g., a subject suffering from a disease as disclosed herein).

Several embodiments relate to a stable crystalline form or the salts or stable crystalline salt forms or two chiral isomers (Compound R-A, Compound S-A) of 5-(2-(4-(4-fluoro-2-methyl-1H-indol-5-yloxy)-6-methoxyquinolin-7-yloxy)ethyl)-5-azaspiro[2.4]-heptan-7-ol for a method in mono therapy or in combination with immunotherapy agents. In several embodiments, the immunotherapy agent is selected from PD-1, PD-L1, oncolytic virus therapy, bispecific T cell engagers (BiTE) and chimeric antigen receptor (CAR) T cell therapy based reagents which including but not limited to nivolumab, pembrolizumab, ipilimumab, blinatumomab, elotuzumab, daratumumab, cemiplimab, avelumab, durvalumab, atezolizumab, toripalimab, sintilimab, camrelizumab, tislelizumab, AK104, Penpulimab, KN035, CS1001, talimogene laherparepvec. In several embodiments, the combination is used to treat a disease or disorder as disclosed elsewhere herein. For example, in treating solid tumors, selected from lung, renal, colorectal, gastric, melanoma, head/neck, thyroid, pancreatic, liver, prostate, bladder, brain, sarcoma, breast, ovarian, cervical and endometrial cancers; and blood cancers, selected from ALL, CLL, AML, CML and Multiple Myeloma.

To test the biological activity, the PTK inhibition activities of Compound A or Compound A-Xln was compared with a market drug Sunitinib. In vitro tyrosine kinase inhibition activities can be measured via commercial resources. Some of these tests can also be contracted with Eurofin or Reaction Biology for screening.

For the kinase inhibition tests, receptor tyrosine kinases are selected from FGFR1(h), FGFR2(h), FGFR3(h), Flt1(h) (VEGFr1), Flt4(h) (VEGFr3), KDR(h) (VEGFr2), Aurora-B, PDGFRα(h) and PDGFRβ(h). Compounds A shows inhibition to all targets and the IC50 value range from sub-nanomole to micromole scale. Comparing with the market drug Sunitinib, Compound A shows higher efficacy of inhibition.

The inhibitions of Compound A-Xln, Compound A-P-Xln and two chiral isomers (Compound R-A, Compound S-A) to cancer cell lines are tested via in vitro MTT assay. Cancer cell lines are selected from PANC-1, NCI-H157, MDA-MB-231, Hela, PC-3, BEL7404, MKN45, Ishikawa, Saos-2, SKOV3, SW579 and HCT116 cell lines. The IC50 values are in the micromole scale.

The following examples further illustrate the present invention but should not be construed as in any way to limit its scope.

### EXAMPLES

### Abbreviations and Definitions

The following abbreviations are used and have the meaning below for ease of reference. EtOH: ethanol, MeOH: methanol, IPA: isopropanol, EtOAc: ethyl acetate, DCM: Dichloromethane, DMF: N,N-dimethylformamide, DMA: N,N-dimethylacetamide, NMP: N-methyl-2-pyrrolidone, RT: room temperature, Temp: temperature, eq: equivalent, g: gram, mg: milligram, ml: milliliter, min: minutes. API: active pharmaceutical ingredient. DSC: differential scanning calorimetric, TGA: thermogravimetric analysis, XRPD: X-ray powder diffraction, Exo: exotherm, Endo: endotherm. ALL: Acute Lymphocytic or Lymphoblastic Leukemias, CLL: Chronic Lymphocytic or Lymphoblastic Leukemias, AML: Acute Myelogenous or Myeloid Leukemias, CML: Chronic Myelogenous or Myeloid Leukemias, NSCLC: non-small cell lung cancer, SCLC: small cell lung cancer, ULMS: Uterine Leiomyosarcoma (Sarcoma), OC: Ovarian Cancer, BT: Brain Tumor, SD: stable disease, PR: partial response, PFS: Progression Free Survival, IC50: half maximal inhibitory concentration, which is the concentration of inhibitor in the cell culture medium, required to inhibit a protein's transporting activity by 50%. MTT: 3-(4,5-dimethylthiazol-2-yl)-2,5-diphenyltetrazolium bromide.

### Example 1: The Preparation of Crystalline Form 5-(2-(4-(4-fluoro-2-methyl-1H-indol-5-yloxy)-6-methoxyquinolin-7-yloxy)ethyl)-5-azaspiro[2.4]-heptan-7-ol (Compound A-Xln)

To the crude Compound A (18 g) similarly prepared according to WO2010021918 to give an amorphous solid (Compound A-Amp) with major peak of DSC at 245.99° C. of Compound A. It was added DMF (180 ml) and the mixture was stirred at 120° C. until the solid dissolved. The hot solution was filtered and the filtrate was concentrated under vacuum to about half volume and further cooled to RT. The precipitate was filtered and the filter cake was washed with small amount of DMF followed by some water. The filter cake was then mixed with ethanol (4 l) and heated to dissolve. The solution was concentrated in vacuum to about half volume and further cooled to RT. The precipitate was filtered and dried in an oven to afford the stable crystal (Compound A-Xln) as desired product with only one peak of DSC at 248.79° C.

DSC graph for amorphous form (Compound A-Amp) of Compound A is represented in the drawing section as FIG. 1.

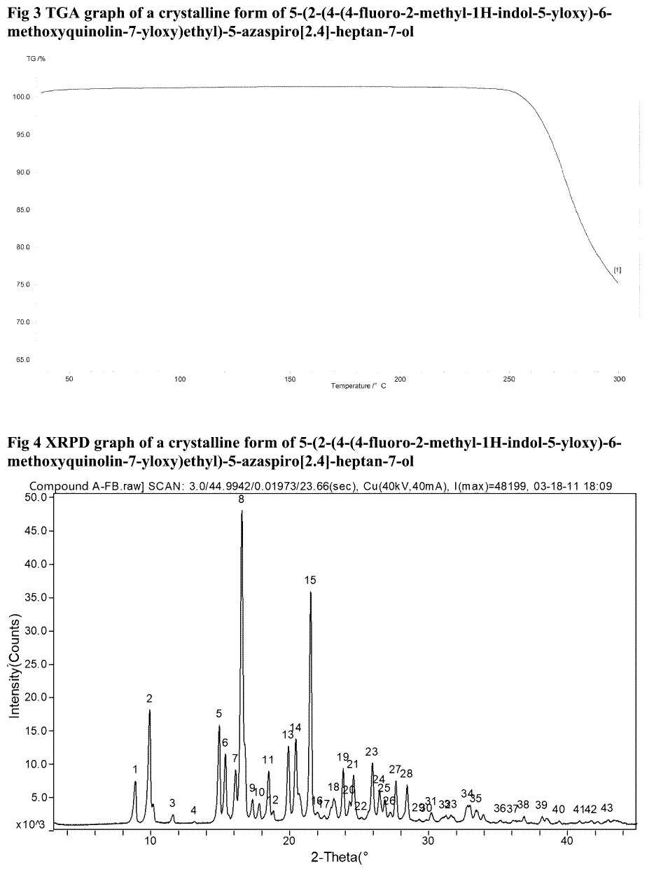

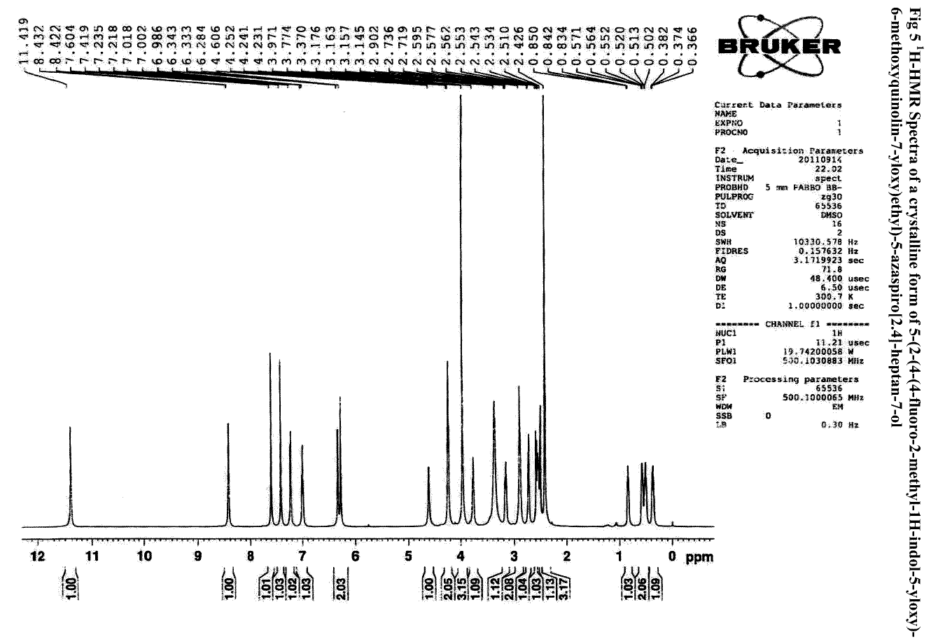

DSC, TGA, XRPD and ¹H-NMR graphs for a crystalline form (Compound A-Xln) of Compound A are represented in the drawing section as FIG. 2, FIG. 3, FIG. 4 and FIG. 5, respectively.

MS: (M+H⁺)/z 478;

¹H-NMR (DMSO-d₆) δ ppm: 0.37-0.39 (m, 1H), 0.50-0.57 (m, 2H), 0.82-0.85 (m, 1H) 2.43 (s, 3H), 2.54-2.59 (d, 2H), 3.11-3.13 (d, 1H), 3.26 (s, 2H), 3.42-3.46 (m, 1H), 3.82-3.84 (t, 1H), 3.98 (s, 3H), 4.39 (t, 2H), 6.28 (s, 2H), 6.35-6.36 (d, 1H), 6.98-7.02 (t, 1H), 7.22-7.24 (d, 1H), 7.48 (s, 1H), 7.62 (s, 1H), 8.43-8.46 (d, 1H), 11.42 (s, 1H).

DSC Melting Range (Endo): 247-253° C. with Peak Temp=248.79° C. TGA demonstrating as an unsolvated material with weight loss above 250° C. XRPD having pattern compromising characteristic 14 peaks with intensity % greater than 10% or 43 characteristic peaks with all intensity % expressed in d values and angles as follows:

### XRPD Data:

| NO. | Angle | d value | Intensity | Intensity % |
|---|---|---|---|---|
| 1 | 8.899 | 9.9286 | 74540 | 12.04 |
| 2 | 9.927 | 8.9032 | 235708 | 38.08 |
| 3 | 11.586 | 7.6312 | 14908 | 2.41 |
| 4 | 13.145 | 6.7299 | 3511 | 0.57 |
| 5 | 14.938 | 5.9257 | 159717 | 25.80 |
| 6 | 15.374 | 5.7587 | 104484 | 16.88 |
| 7 | 16.104 | 5.4991 | 93019 | 15.03 |
| 8 | 16.558 | 5.3495 | 619004 | 100.00 |
| 9 | 17.327 | 5.1137 | 22260 | 3.60 |
| 10 | 17.820 | 4.9734 | 20322 | 3.28 |
| 11 | 18.492 | 4.7942 | 87559 | 14.15 |
| 12 | 18.809 | 4.7140 | 21872 | 3.53 |
| 13 | 19.912 | 4.4553 | 118882 | 19.21 |
| 14 | 20.445 | 4.3403 | 180671 | 29.19 |
| 15 | 21.511 | 4.1275 | 348507 | 56.30 |
| 16 | 22.004 | 4.0362 | 14250 | 2.30 |
| 17 | 22.499 | 3.9485 | 3195 | 0.52 |
| 18 | 23.188 | 3.8327 | 51679 | 8.35 |
| 19 | 23.859 | 3.7265 | 69270 | 11.19 |
| 20 | 24.314 | 3.6577 | 67756 | 10.95 |
| 21 | 24.590 | 3.6172 | 93287 | 15.07 |
| 22 | 25.171 | 3.5350 | 1707 | 0.28 |
| 23 | 25.952 | 3.4305 | 86263 | 13.94 |
| 24 | 26.464 | 3.3652 | 53165 | 8.59 |
| 25 | 26.841 | 3.3188 | 20232 | 3.27 |
| 26 | 27.217 | 3.2738 | 6582 | 1.06 |
| 27 | 27.648 | 3.2237 | 46760 | 7.55 |
| 28 | 28.457 | 3.1339 | 54339 | 8.78 |
| 29 | 29.310 | 3.0446 | 1984 | 0.32 |
| 30 | 29.856 | 2.9901 | 4264 | 0.69 |
| 31 | 30.212 | 2.9557 | 18700 | 3.02 |
| 32 | 31.202 | 2.8642 | 26858 | 4.34 |
| 33 | 31.632 | 2.8262 | 24054 | 3.89 |
| 34 | 32.837 | 2.7252 | 55773 | 9.01 |
| 35 | 33.430 | 2.6782 | 39092 | 6.32 |
| 36 | 35.185 | 2.5485 | 6426 | 1.04 |
| 37 | 36.092 | 2.4865 | 16774 | 2.71 |
| 38 | 36.865 | 2.4361 | 20105 | 3.25 |
| 39 | 38.167 | 2.3560 | 16104 | 2.60 |
| 40 | 39.409 | 2.2845 | 5045 | 0.82 |
| 41 | 40.888 | 2.2053 | 5238 | 0.85 |
| 42 | 41.720 | 2.1632 | 8720 | 1.41 |
| 43 | 42.886 | 2.1070 | 9964 | 1.61 |

### Example 2

Based on the inventor's experience of research that a crystalline form preparation of 5-(2-(4-(4-fluoro-2-methyl-1H-indol-5-yloxy)-6-methoxyquinolin-7-yloxy)ethyl)-5-azaspiro[2.4]-heptan-7-ol (Compound A) in Example 1, a following crystalline form preparation of Compound A is expected. A crystalline form (Compound A-Xln) preparation of Compound A should contain at least one recrystallization process, preferably two recrystallization processes. Preferably, the initial recrystallization requires a relatively high boiling point solvent (or a mixture of solvents) which includes but not limited to DMF, DMA and NMP. The subsequent recrystallization process requires a kind of solvent (or a mixture of solvents) which includes but not limited to MeOH, EtOH and IPA.

### Example 3: Stability Comparison of Amorphous (Compound A-Amp) and Crystalline Form (Compound A-Xln) API of (2-(4-(4-fluoro-2-methyl-1H-indol-5-yloxy)-6-methoxyquinolin-7-yloxy)ethyl)-5-azaspiro[2.4]-heptan-7-ol (Compound A)

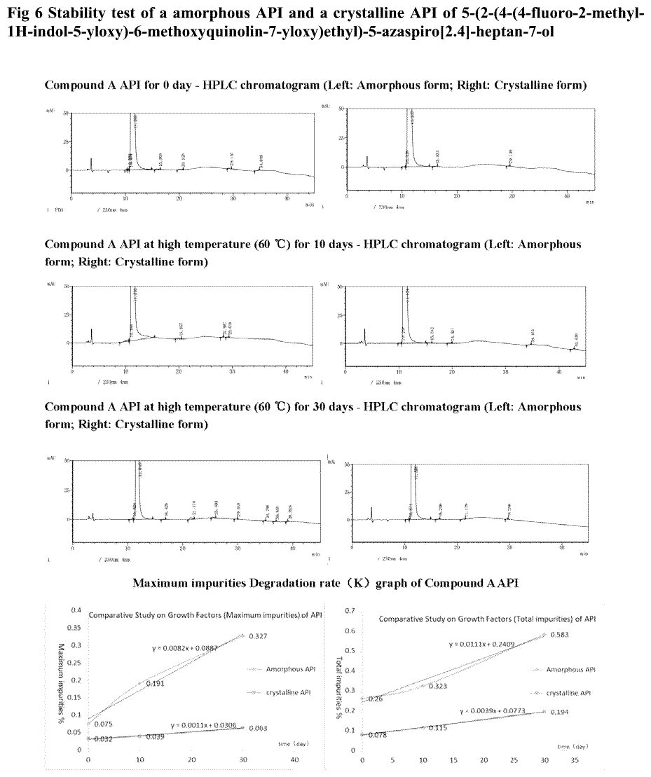

The samples of amorphous and crystalline form API of Compound A were placed in clean containers and the environmental temperature was controlled at 60° C. The purity of amorphous (Compound A-Amp) and crystalline form (Compound A-Xln) of Compound A were tested at Day 0, Day 10 and Day 30. Samples of amorphous and crystalline form APIs were measured, and the appropriate amount of diluent was added to dissolve with sonication. The concentration was controlled at 0.2 mg/ml and the purification was determined by HPLC with UV detector at 230 nm. Results were listed in the table as follows and the fitted curve is in Drawing Sections as FIG. 6. A-Xln has better stability than A-Amp

TABLE 1
Stability comparison of amorphous and crystalline form API of Compound A

| | Relative substances | | | | | | | |
|---|---|---|---|---|---|---|---|---|
| | | | High temperature | | High temperature | | | |
| | 0 day | | 60° C., 10 days | | 60° C., 30 days | | Maximum | Total |
| | Maximum | Total | Maximum | Total | Maximum | Total | impurities | impurities |
| Compound | impurities | impurities | impurities | impurities | impurities | impurities | Degradation | Degradation |
| name | (%) | (%) | (%) | (%) | (%) | (%) | rate (K) | rate (K) |
| (Compound | 0.075 | 0.26 | 0.191 | 0.323 | 0.327 | 0.583 | 0.0082 | 0.0111 |
| A-Amp) | | | | | | | | |
| API | | | | | | | | |
| (Compound | 0.032 | 0.078 | 0.039 | 0.115 | 0.063 | 0.194 | 0.0011 | 0.0039 |
| A-Xln) API | | | | | | | | |

### Example 4: Stability Comparison of Amorphous (Compound A-Amp) and Crystalline Form (Compound A-Xln) Tablets of (2-(4-(4-fluoro-2-methyl-1H-indol-5-yloxy)-6-methoxyquinolin-7-yloxy)ethyl)-5-azaspiro[2.4]-heptan-7-ol (Compound A)

The sample of amorphous and crystalline form tablets (10 mg or 30 mg) of Compound A (A-Amp or A-Xln; named Compound A-Amp tablet, or Compound A-Xln tablet) were prepared in following process with the listed tablet composition below:
(1) Sieve process: Compound A (A-Amp or A-Xln), lactose T80, microcrystalline cellulose (PH101), Sodium Carboxymethyl Starch, were sieved through 80-mesh screen, respectively.
(2) Granulation process: The sieved Compound A (A-Amp or A-Xln), lactose T80, microcrystalline cellulose, sodium carboxymethyl starch and silicon dioxide were charged into a V-shape blender for blending 1. After the blending 1, the material was fed to a roller compactor for dry granulation. The dry granules were sieved through a 24-mesh screen and the resulting powder was sieved through a 50-mesh screen to provide desired fine powder, which counts for 10% to 13% of the total particle weight. The sieved granules were blended with pre-sieved, magnesium stearate for blending 2, and the content of the final blend was sampled and tested for blend assay (8.9% to 10.3%).
(3) Tableting process: After setting up the Tablet Press for compression into 10 mg and 30 mg, the final blend was compressed into tablets. In-process control (IPC) tests were performed during the processing for weight variation and hardness (Weight variation is ±6.0% and hardness is 7 to 13 kg/mm²).
(4) Coating process: the coating solution was prepared according to the ratio of gastric-soluble film coating premix: purified water=15:85 (w:w), and keep stirring for 45 minutes. The coating weight gain was about 2% to 3%.
(5) Packaging process: The collected tablets were processed with weight sorting. The accepted tablets were collected and packaged in Aluminum-plastic blister with 7 tablets/board.

TABLE 2
Composition of Compound A Drug Product (10 mg Tablet)

| | Quality | | | Dosage |
|---|---|---|---|---|
| Component | Reference | Function | wt. % | (mg) |
| Compound A (A-Amp or A-Xln) | In-house | Active | 9.24 | 10 |
| | | ingredient | | |
| Lactose T80 | Chp/USP | Filler | 55.42 | 60 |
| Microcrystalline cellulose (PH101) | Chp/USP | Filler | 27.71 | 30 |
| Sodium Carboxymethyl Starch | Chp/USP | Disintegrant | 3.69 | 4 |
| Silicon dioxide | Chp/USP | Lubricant | 7.11 | 0.77 |
| Magnesium Stearate | Chp/USP | Lubricant | 0.46 | 0.5 |
| Opadry gastric-soluble coating powder | Chp/USP | Coating | 2.77 | 3 |

| | | |
|---|---|---|
| Total | 100.0 | 108.27 |

Chp: Chinese Pharmacopoeia;
USP: United States of Pharmacopoeia

TABLE 3
Composition of Compound A Drug Product (30 mg Tablet)

| | Quality | | | Dosage |
|---|---|---|---|---|
| Component | Reference | Function | wt. % | (mg) |
| Compound A (A-Amp or A-Xln) | In-house | Active | 9.24 | 30 |
| | | ingredient | | |
| Lactose T80 | Chp/USP | Filler | 55.42 | 180 |
| Microcrystalline cellulose (PH101) | Chp/USP | Filler | 27.71 | 90 |
| Sodium Carboxymethyl Starch | Chp/USP | Disintegrant | 3.69 | 12 |
| Silicon dioxide | Chp/USP | Lubricant | 7.11 | 2.31 |
| Magnesium Stearate | Chp/USP | Lubricant | 0.46 | 1.5 |
| Opadry gastric-soluble coating powder | Chp/USP | Coating | 2.77 | 9 |

| | | |
|---|---|---|
| Total | 100.0 | 324.81 |

Chp: Chinese Pharmacopoeia;
USP: United States of Pharmacopoeia

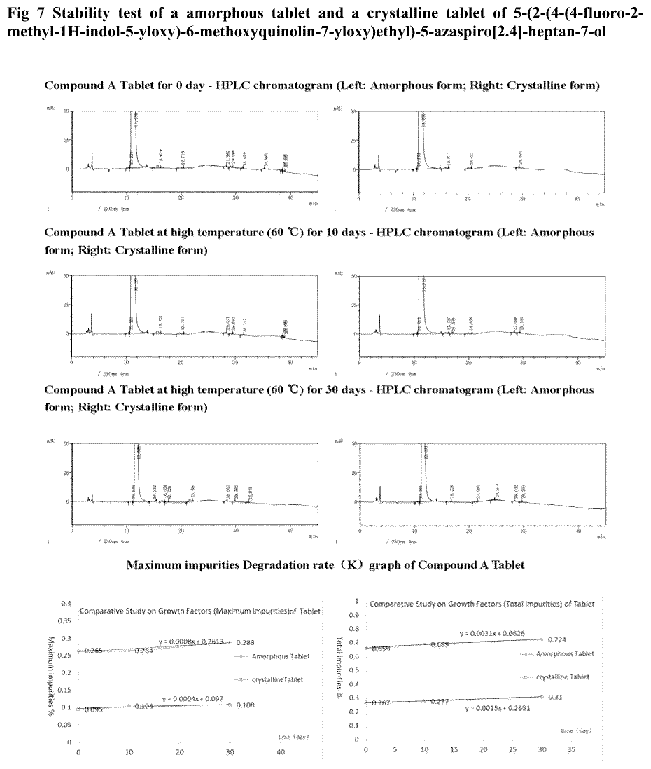

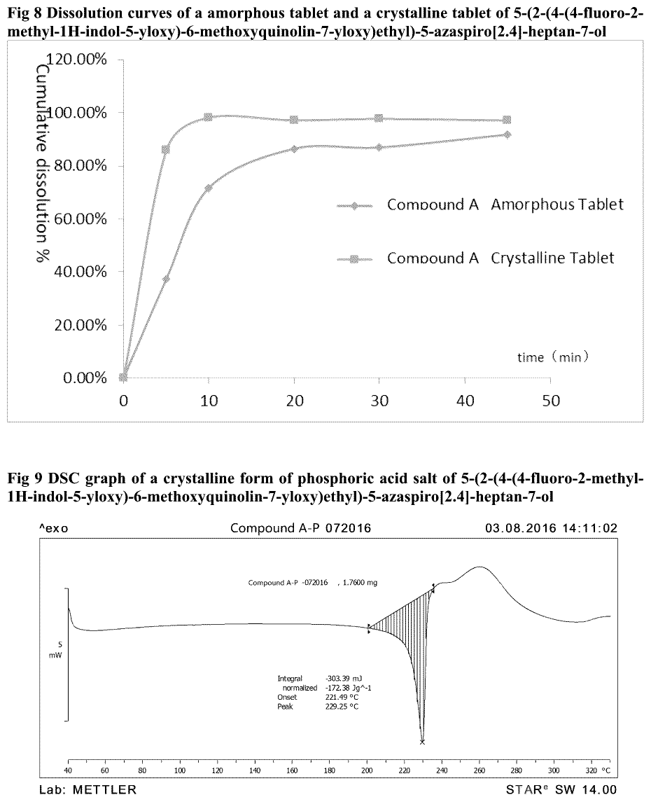

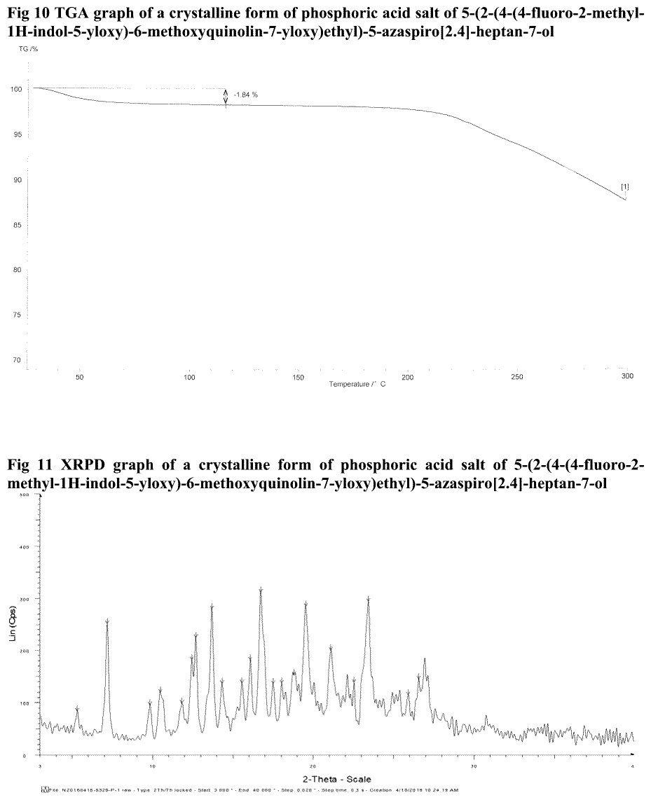

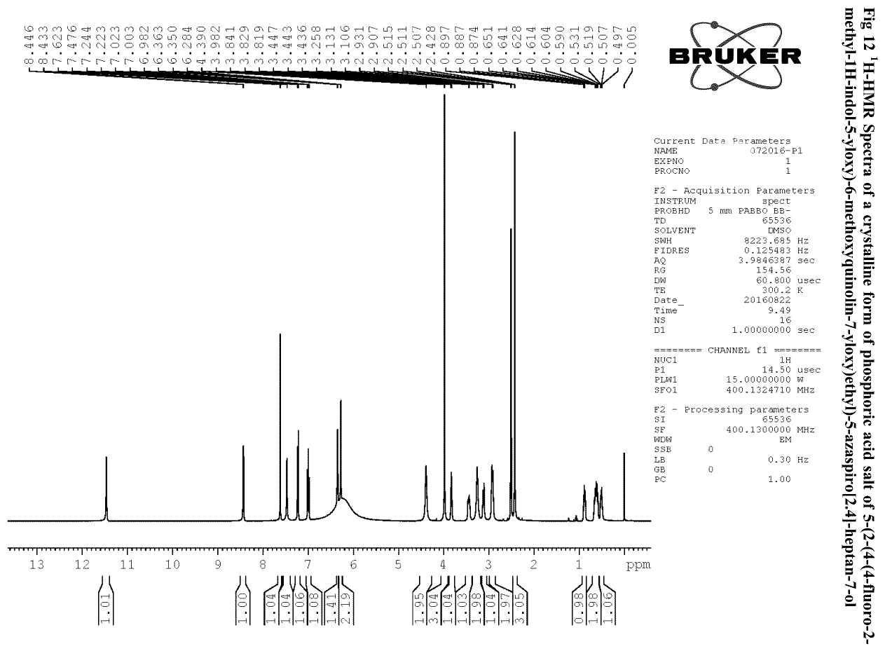

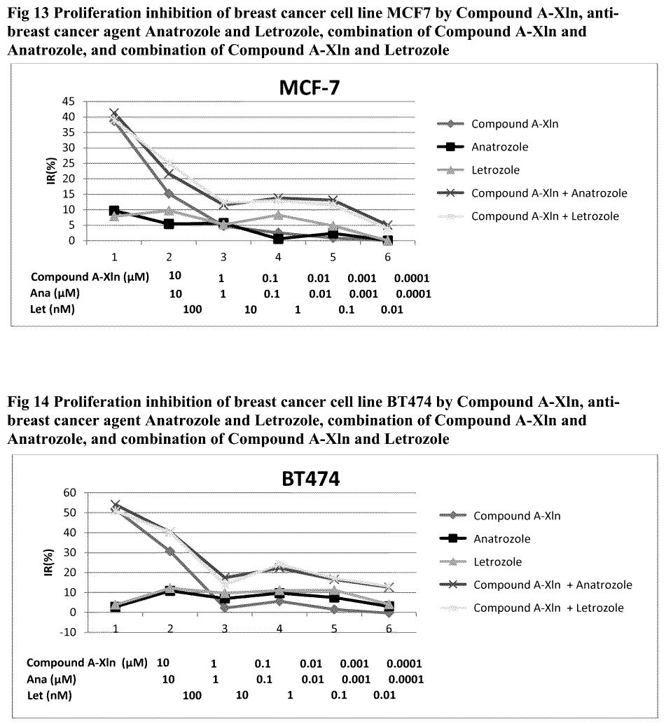

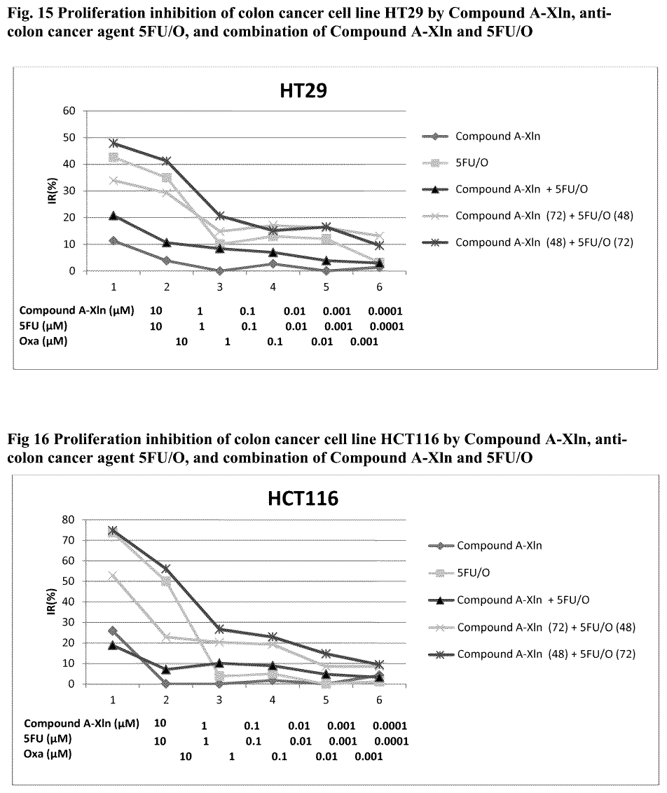

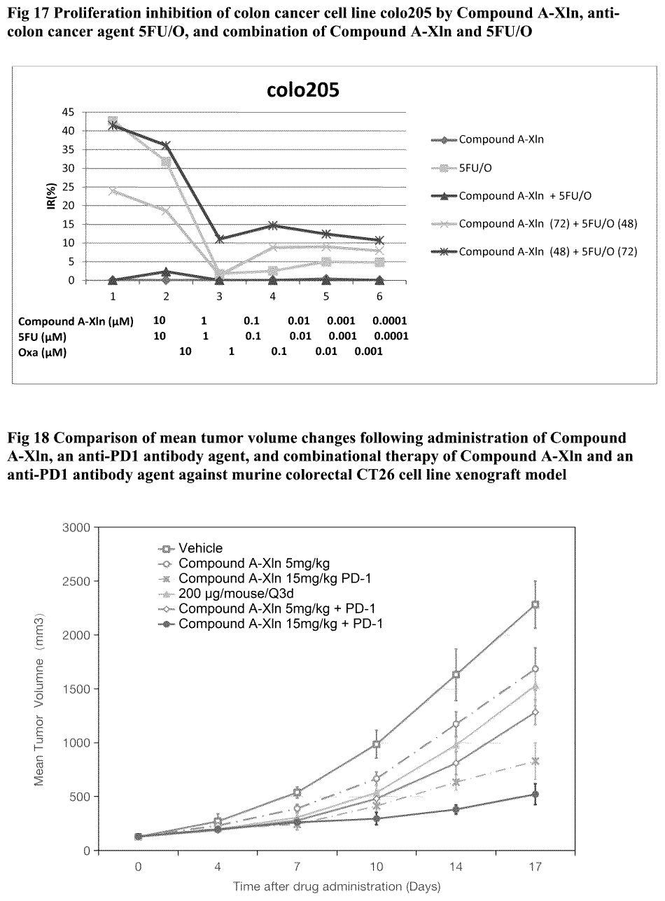

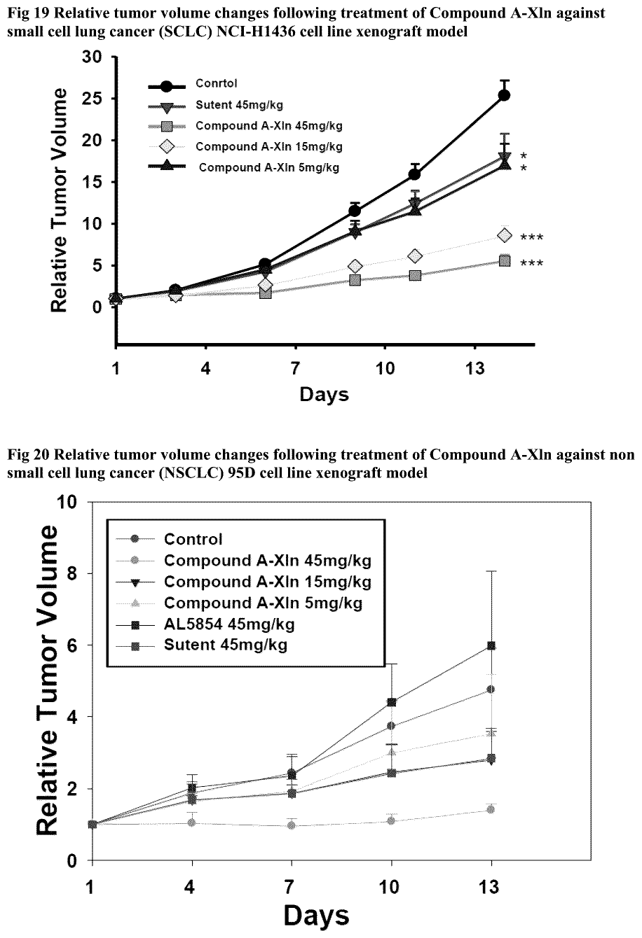

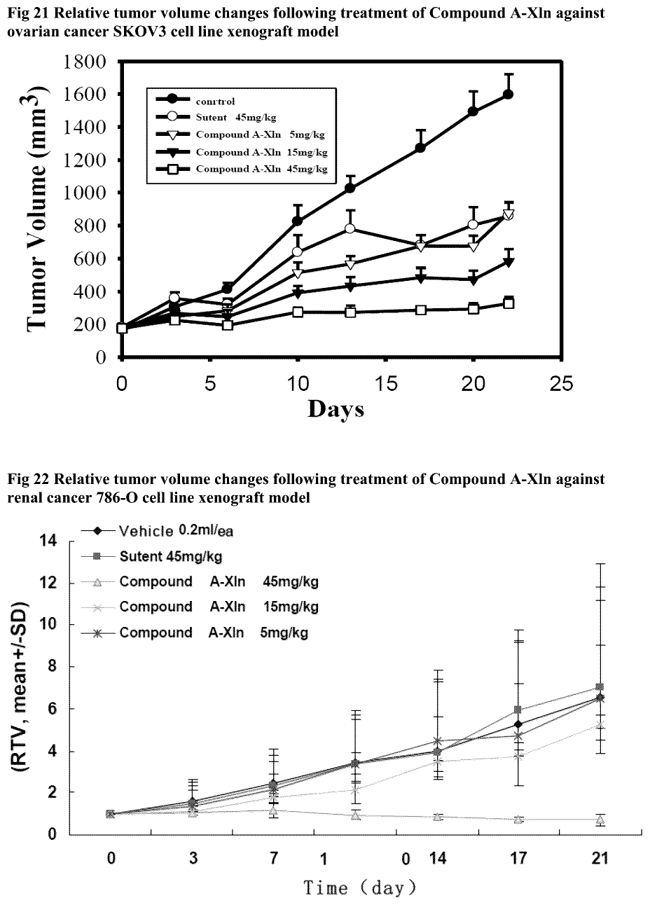

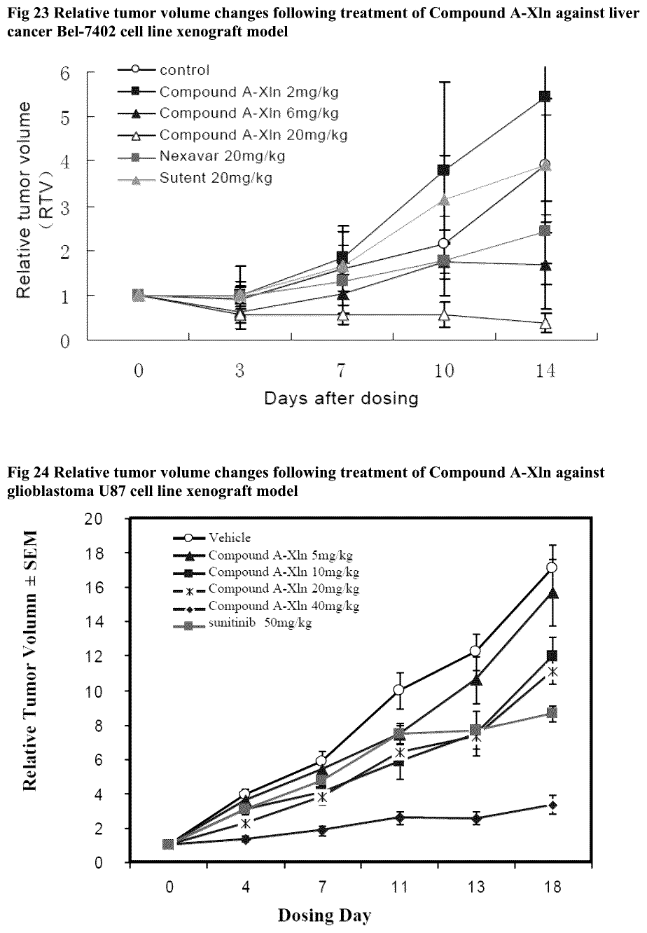

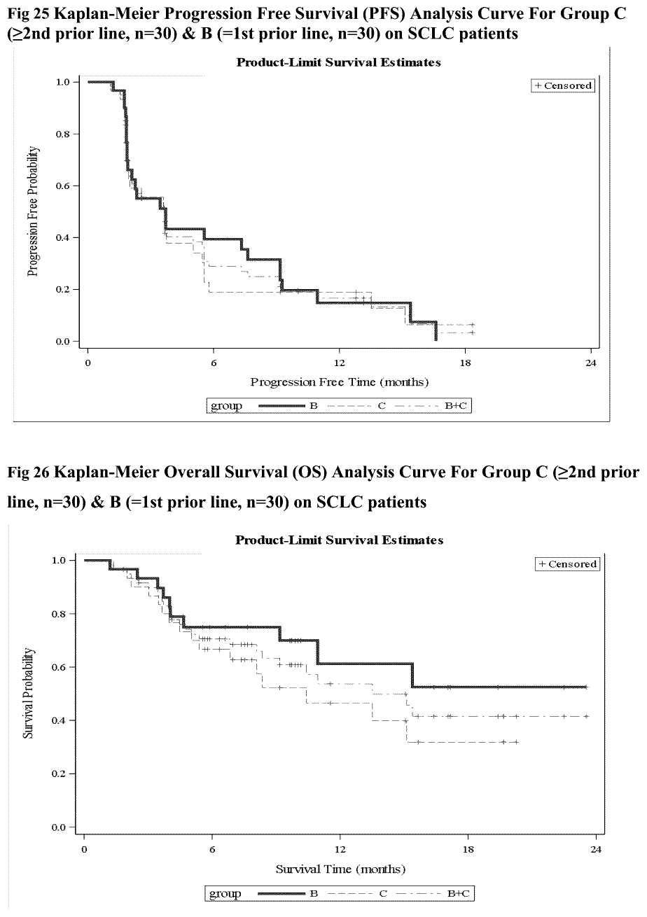

The prepared tablets were tested in the similar protocol as described in Example 3 and the results were listed in Table 4 and the fitted curve is in Drawing Sections as FIG. 7.

TABLE 4
Stability comparison of amorphous and crystalline form Tablets of Compound A

| | Relative substance | | | | | | | |
|---|---|---|---|---|---|---|---|---|
| | | | High temperature | | High temperature | | | |
| | 0 day | | 60° C., 10 days | | 60° C., 30 days | | Maximum | Total |
| | maximum | Total | maximum | Total | Maximum | Total | impurities | impurities |
| Compound | impurities | impurities | impurities | impurities | impurities | impurities | Degradation | Degradation |
| name | (%) | (%) | (%) | ( %) | (%) | ( %) | rate (K) | rate (K) |
| Compound | 0.265 | 0.659 | 0.264 | 0.689 | 0.288 | 0.724 | 0.0008 | 0.0021 |
| A-Amp | | | | | | | | |
| Tablet | | | | | | | | |
| Compound | 0.095 | 0.267 | 0.104 | 0.277 | 0.108 | 0.31 | 0.0004 | 0.0015 |
| A-Xln | | | | | | | | |
| Tablet | | | | | | | | |

TABLE 5
Stability of Compound A-XIn Drug Product (DP, tablets), Stored at 25° C. ± 2° C./60%
RH ± 5% RH

| | | | | | Related Substance | | |
|---|---|---|---|---|---|---|---|
| | Study | | | | Maximum | | |
| Batch | initiation | Timepoint | | | single | Total | |
| # | Date | (Month) | Appearance | Dissolution | impurity | Impurities | Assay |
| Strength | | | 10 mg | | | | |
| Specifications | | | Film-coated tablets, Remove the | 75% of the | ≤0.5% | ≤1.0% | 90.0%- |
| | | | film coating show off- white. | labeled | | | 110.0% |
| 20120801 | Jan. | 0 | Film-coated tablets, Remove the | 94.01 | 0.15 | 0.67 | 106.19 |
| | 15, 2013 | | film coating show off-white. | | | | |
| | | 3 | Film-coated tablets, Remove the | 104.88 | 0.12 | 0.57 | 106.80 |
| | | | film coating show off-white. | | | | |
| | | 6 | Film-coated tablets, Remove the | 99.08 | 0.16 | 0.71 | 101.25 |
| | | | film coating show off-white. | | | | |
| | | 9 | Film-coated tablets, Remove the | 84.07 | 0.16 | 0.78 | 107.74 |
| | | | film coating show off-white. | | | | |
| | | 12 | Film-coated tablets, Remove the | 94.45 | 0.17 | 0.82 | 105.68 |
| | | | film coating show off-white. | | | | |
| | | 18 | Film-coated tablets, Remove the | 88.13 | 0.22 | 0.77 | 108.93 |
| | | | film coating show off-white. | | | | |
| | | 24 | Film-coated tablets, Remove the | 105.87 | 0.29 | 0.97 | 101.91 |
| | | | film coating show off-white. | | | | |
| | | 36 | Film-coated tablets, Remove the | 99.15 | 0.30 | 0.91 | 103.49 |
| | | | film coating show off-white. | | | | |
| 20120802 | Jan. | 0 | Film-coated tablets, Remove the | 96.55 | 0.10 | 0.54 | 106.02 |
| | 15, 2013 | | film coating show off-white. | | | | |
| | | 3 | Film-coated tablets, Remove the | 96.32 | 0.14 | 0.62 | 106.06 |
| | | | film coating show off-white. | | | | |
| | | 6 | Film-coated tablets, Remove the | 96.98 | 0.17 | 0.86 | 102.06 |
| | | | film coating show off-white. | | | | |
| | | 9 | Film-coated tablets, Remove the | 83.31 | 0.17 | 0.93 | 104.18 |
| | | | film coating show off-white. | | | | |
| | | 12 | Film-coated tablets, Remove the | 109.99 | 0.23 | 0.88 | 107.04 |
| | | | film coating show off-white. | | | | |
| | | 18 | Film-coated tablets, Remove the | 83.87 | 0.24 | 0.71 | 105.22 |
| | | | film coating show off-white. | | | | |
| | | 24 | Film-coated tablets, Remove the | 101.57 | 0.43 | 0.88 | 99.08 |
| | | | film coating show off-white. | | | | |
| | | 36 | Film-coated tablets, Remove the | 102.26 | 0.33 | 0.91 | 105.91 |
| | | | film coating show off-white. | | | | |
| 20120803 | Jan. | 0 | Film-coated tablets, Remove the | 99.07 | 0.18 | 0.72 | 107.43 |
| | 15, 2013 | | film coating show off-white. | | | | |
| | | 3 | Film-coated tablets, Remove the | 100.15 | 0.14 | 0.75 | 106.36 |
| | | | film coating show off-white. | | | | |
| | | 6 | Film-coated tablets, Remove the | 104.01 | 0.20 | 0.89 | 103.74 |
| | | | film coating show off-white. | | | | |
| | | 9 | Film-coated tablets, Remove the | 92.97 | 0.21 | 0.89 | 104.20 |
| | | | film coating show off-white. | | | | |
| | | 12 | Film-coated tablets, Remove the | 98.62 | 0.21 | 0.79 | 107.95 |
| | | | film coating show off-white. | | | | |
| | | 18 | Film-coated tablets, Remove the | 89.56 | 0.27 | 1.04 | 107.26 |
| | | | film coating show off-white. | | | | |
| | | 24 | Film-coated tablets, Remove the | 103.27 | 0.40 | 0.85 | 98.92 |
| | | | film coating show off-white. | | | | |
| | | 36 | Film-coated tablets, Remove the | 102.75 | 0.38 | 1.12 | 106.25 |
| | | | film coating show off-white. | | | | |
| 2016050110 | Jun. 14, | 0 | Film-coated tablets, Remove the | 99.26 | 0.08 | 0.31 | 97.60 |
| | 2016 | | film coating show off-white. | | | | |
| | | 3 | Film-coated tablets, Remove the | 93.40 | 0.08 | 0.39 | 99.57 |
| | | | film coating show off-white. | | | | |
| | | 6 | Film-coated tablets, Remove the | 94.06 | 0.08 | 0.42 | 94.02 |
| | | | film coating show off-white. | | | | |
| | | 9 | Film-coated tablets, Remove the | 90.82 | 0.27 | 0.64 | 102.74 |
| | | | film coating show off-white. | | | | |
| | | 12 | Film-coated tablets, Remove the | 90.81 | 0.15 | 0.52 | 98.22 |
| | | | film coating show off-white. | | | | |
| | | 18 | Film-coated tablets, Remove the | 93.26 | 0.18 | 0.57 | 99.85 |
| | | | film coating show off-white. | | | | |
| | | 24 | Film-coated tablets, Remove the | 96.70 | 0.17 | 0.76 | 94.84 |
| | | | film coating show off-white. | | | | |
| 2016050210 | Jun. | 0 | Film-coated tablets, Remove the | 92.51 | 0.11 | 0.32 | 98.36 |
| | 14, 2016 | | film coating show off-white. | | | | |
| | | 3 | Film-coated tablets, Remove the | 91.31 | 0.14 | 0.38 | 98.23 |
| | | | film coating show off-white. | | | | |
| | | 6 | Film-coated tablets, Remove the | 92.51 | 0.08 | 0.39 | 94.05 |
| | | | film coating show off-white. | | | | |
| | | 9 | Film-coated tablets, Remove the | 89.55 | 0.17 | 0.52 | 101.55 |
| | | | film coating show off-white. | | | | |
| | | 12 | Film-coated tablets, Remove the | 90.12 | 0.11 | 0.54 | 98.35 |
| | | | film coating show off-white. | | | | |
| | | 18 | Film-coated tablets, Remove the | 85.90 | 0.20 | 0.64 | 96.03 |
| | | | film coating show off-white. | | | | |
| | | 24 | Film-coated tablets, Remove the | 97.94 | 0.22 | 0.70 | 90.29 |
| | | | film coating show off-white. | | | | |
| 2016050310 | Jun. | 0 | Film-coated tablets, Remove the | 101.86 | 0.11 | 0.32 | 97.37 |
| | 14, 2016 | | film coating show off-white. | | | | |
| | | 3 | Film-coated tablets, Remove the | 93.20 | 0.14 | 0.41 | 99.90 |
| | | | film coating show off-white. | | | | |
| | | 6 | Film-coated tablets, Remove the | 98.81 | 0.09 | 0.36 | 95.10 |
| | | | film coating show off-white. | | | | |
| | | 9 | Film-coated tablets, Remove the | 92.31 | 0.20 | 0.58 | 101.20 |
| | | | film coating show off-white. | | | | |
| | | 12 | Film-coated tablets, Remove the | 95.21 | 0.17 | 0.63 | 98.01 |
| | | | film coating show off-white. | | | | |
| | | 18 | Film-coated tablets, Remove the | 92.57 | 0.21 | 0.70 | 94.84 |
| | | | film coating show off-white. | | | | |
| | | 24 | Film-coated tablets, Remove the | 91.17 | 0.27 | 0.87 | 94.22 |
| | | | film coating show off-white. | | | | |
| Specifications | | | Film-coated tablets, Remove the | 75% of | ≤0.5% | ≤1.0% | 90.0%- |
| | | | film coating show off- white. | the labeled | | | 110.0% |
| | | | | amount | | | |
| Strength | | | 30 mg | | | | |
| 2016050130 | Jun. 14, | 0 | Film-coated tablets, Remove the | 98.46 | 0.09 | 0.32 | 96.47 |
| | 2016 | | film coating show off-white. | | | | |
| | | 3 | Film-coated tablets, Remove the | 93.50 | 0.10 | 0.41 | 100.15 |
| | | | film coating show off-white. | | | | |
| | | 6 | Film-coated tablets, Remove the | 93.69 | 0.10 | 0.37 | 93.96 |
| | | | film coating show off-white. | | | | |
| | | 9 | Film-coated tablets, Remove the | 90.72 | 0.26 | 0.64 | 100.79 |
| | | | film coating show off-white. | | | | |
| | | 12 | Film-coated tablets, Remove the | 94.62 | 0.15 | 0.61 | 97.71 |
| | | | film coating show off-white. | | | | |
| | | 18 | Film-coated tablets, Remove the | 94.80 | 0.20 | 0.61 | 95.32 |
| | | | film coating show off-white. | | | | |
| | | 24 | Film-coated tablets, Remove the | 94.42 | 0.25 | 0.81 | 94.29 |
| | | | film coating show off-white. | | | | |
| 2016050230 | Jun. 14, | 0 | Film-coated tablets, Remove the | 100.02 | 0.10 | 0.31 | 97.64 |
| | 2016 | | film coating show off-white. | | | | |
| | | 3 | Film-coated tablets, Remove the | 93.87 | 0.09 | 0.42 | 102.54 |
| | | | film coating show off-white. | | | | |
| | | 6 | Film-coated tablets, Remove the | 97.54 | 0.10 | 0.36 | 96.47 |
| | | | film coating show off-white. | | | | |
| | | 9 | Film-coated tablets, Remove the | 94.50 | 0.19 | 0.50 | 101.41 |
| | | | film coating show off-white. | | | | |
| | | 12 | Film-coated tablets, Remove the | 96.99 | 0.16 | 0.68 | 98.13 |
| | | | film coating show off-white. | | | | |
| | | 18 | Film-coated tablets, Remove the | 92.57 | 0.20 | 0.65 | 97.26 |
| | | | film coating show off-white. | | | | |
| | | 24 | Film-coated tablets, Remove the | 93.13 | 0.20 | 0.77 | 95.63 |
| | | | film coating show off-white. | | | | |
| 2016050330 | Jun. 14, | 0 | Film-coated tablets, Remove the | 96.10 | 0.09 | 0.29 | 96.53 |
| | 2016 | | film coating show off-white. | | | | |
| | | 3 | Film-coated tablets, Remove the | 97.44 | 0.07 | 0.39 | 102.33 |
| | | | film coating show off-white. | | | | |
| | | 6 | Film-coated tablets, Remove the | 103.30 | 0.10 | 0.41 | 95.16 |
| | | | film coating show off-white. | | | | |
| | | 9 | Film-coated tablets, Remove the | 88.08 | 0.20 | 0.55 | 100.11 |
| | | | film coating show off-white. | | | | |
| | | 12 | Film-coated tablets, Remove the | 92.58 | 0.09 | 0.46 | 97.29 |
| | | | film coating show off-white. | | | | |
| | | 18 | Film-coated tablets, Remove the | 92.47 | 0.15 | 0.49 | 94.74 |
| | | | film coating show off-white. | | | | |
| | | 24 | Film-coated tablets, Remove the | 97.69 | 0.24 | 0.97 | 94.40 |
| | | | film coating show off-white. | | | | |

### Example 5: Comparison of in Vitro Dissolution Curves of Compound A Tablets

The crystal form of a compound can change its physical and chemical properties including the dissolution speed that can affect the effectiveness of a drug. The dissolution speed measurement could be accomplished in dissolution tests. Samples of amorphous (Compound A-Amp) tablets and crystalline (Compound A-Xln) tablets were dissolved in an appropriate solvent (0.1 mol hydrochloric acid solution) and being stirred at appropriate speed to facilitate the dissolution process (ZRC-8D intelligent dissolution apparatus at the speed of 50 rpm). The dissolution percentage was determined by HPLC with the UV detector wavelength set at 230 nm. Samples were collected at 5 min, 10 min, 20 min, 30 min and 45 min, the experimental results were listed in the tables as below and the dissolution curves can be located in Drawing Sections as FIG. 8. After the comparison of experimental data, it can be observed that the crystalline tablet dissolves faster than the amorphous tablet.

TABLE 6
Compound A-Amp Tablet 10 mg Specification Dissolution Curve Results

| Time/ | | | | | | | | |
|---|---|---|---|---|---|---|---|---|
| min | 1 | 2 | 3 | 4 | 5 | 6 | AVG | RSD |
| 5 | 35.26% | 40.19% | 38.57% | 34.47% | 36.59% | 38.55% | 37.27% | 5.90% |
| 10 | 70.22% | 69.35% | 72.56% | 70.48% | 71.69% | 75.44% | 71.62% | 3.05% |
| 20 | 83.54% | 82.06% | 86.68% | 91.49% | 86.16% | 88.32% | 86.37% | 3.90% |
| 30 | 84.00% | 82.52% | 87.16% | 91.99% | 86.63% | 88.81% | 86.85% | 3.90% |
| 45 | 94.43% | 94.34% | 87.43% | 91.14% | 91.42% | 91.22% | 91.66% | 2.81% |

TABLE 7
Compound A-Xln Tablet 10 mg Specification Dissolution Curve Results

| Time/ | | | | | | | | |
|---|---|---|---|---|---|---|---|---|
| min | 1 | 2 | 3 | 4 | 5 | 6 | AVG | RSD |
| 5 | 88.79% | 88.69% | 82.24% | 80.02% | 85.03% | 89.94% | 85.79% | 4.69% |
| 10 | 100.99% | 98.14% | 95.45% | 98.40% | 97.32% | 98.87% | 98.19% | 1.86% |
| 20 | 98.82% | 95.70% | 98.58% | 97.03% | 95.58% | 97.27% | 97.16% | 1.41% |
| 30 | 99.36% | 96.23% | 99.13% | 97.56% | 96.10% | 97.80% | 97.70% | 1.41% |
| 45 | 103.53% | 96.06% | 96.95% | 93.98% | 96.03% | 95.91% | 97.08% | 3.41% |

AVG: average value;
RSD: relative standard deviation.

### Example 6: Phosphoric Acid Salt and Crystalline Form (Compound A-P-Xln) Preparation of 5-(2-(4-(4-fluoro-2-methyl-1H-indol-5-yloxy)-6-methoxyquinolin-7-yloxy)ethyl)-5-azaspiro[2.4]-heptan-7-ol

Compound A (250 mg) was dissolved in EtOH (110 ml) at reflux and to the resulting solution was added 1M H₃PO₄ in EtOH (0.53 ml). The reaction was refluxed for 1 hour and it was cooled to RT with slow stirring overnight. The solid was filtered and rinsed with EtOH, further dried in an oven at 50° C. for 10 hours to obtain a white solid as the phosphoric acid salt of Compound A with a stable crystalline form (Compound A-P-Xln).

The DSC, TGA, XRPD and ¹H-NMR graphs of the phosphoric acid salt crystalline (Compound A-P-Xln) are represented by FIG. 9, FIG. 10, FIG. 11 and FIG. 12, respectively.

¹H NMR (DMSO-d₆) δ ppm: 0.37-0.39 (m, 1H), 0.50-0.57 (m, 2H), 0.82-0.85 (m, 1H) 2.43 (s, 3H), 2.51-2.56 (m, 1H), 2.58-2.60 (d, 1H), 2.72-2.74 (d, 1H), 2.90 (t, 2H), 3.14-3.18 (m, 1H), 3.77-3.82 (m, 1H), 3.97 (s, 3H), 4.24 (t, 2H), 4.60 (s, 1H), 6.28 (s, 1H), 6.33-6.34 (d, 1H), 7.00 (t, 1H), 7.22-7.24 (d, 1H), 7.42 (s, 1H), 7.60 (s, 1H), 8.42-8.43 (d, 1H), 11.42 (s, 1H).

DSC Melting Range (Endo): 221-235° C. with Peak Temp=229° C. TGA demonstrating a 1.84% weight loss at about 30-60° C. and a significant weight loss above 210° C. XRPD having pattern compromising characteristic 21 peaks with all intensity % expressed in d values and angles as follows:

TABLE 8
XRPD Data for the phosphoric acid salt of Compound A
with a stable crystalline form (Compound A-P-Xln):

| NO. | Angle | d value | Intensity | Intensity % |
|---|---|---|---|---|
| 1 | 5.268 | 16.760 | 83.1 | 26.6 |
| 2 | 7.139 | 12.372 | 251.0 | 80.6 |
| 3 | 9.805 | 9.014 | 94.9 | 30.4 |
| 4 | 10.455 | 8.455 | 119.0 | 38.2 |
| 5 | 11.799 | 7.494 | 98.1 | 31.4 |
| 6 | 12.417 | 7.123 | 181.0 | 58.1 |
| 7 | 12.669 | 6.982 | 225.0 | 72.0 |
| 8 | 13.672 | 6.472 | 280.0 | 89.8 |
| 9 | 14.307 | 6.186 | 136.0 | 43.5 |
| 10 | 15.546 | 5.695 | 137.0 | 44.0 |
| 11 | 16.064 | 5.513 | 181.0 | 58.1 |
| 12 | 16.719 | 5.298 | 312.0 | 100.0 |
| 13 | 17.495 | 5.065 | 135.0 | 43.4 |
| 14 | 18.035 | 4.915 | 136.0 | 43.7 |
| 15 | 18.802 | 4.716 | 153.0 | 48.9 |
| 16 | 19.530 | 4.542 | 285.0 | 91.3 |
| 17 | 21.095 | 4.208 | 200.0 | 64.1 |
| 18 | 22.535 | 3.942 | 138.0 | 44.2 |
| 19 | 23.426 | 3.794 | 294.0 | 94.3 |
| 20 | 25.916 | 3.435 | 114.0 | 36.6 |
| 21 | 26.577 | 3.351 | 144.0 | 46.3 |

### Example 7

Based on the inventor's experience of research that a stable crystalline phosphate salt form (Compound A-P-Xln) preparation of 5-(2-(4-(4-fluoro-2-methyl-1H-indol-5-yloxy)-6-methoxy-quinolin-7-yloxy)ethyl)-5-azaspiro[2.4]-heptan-7-ol (Compound A), the following stable crystalline phosphate salt form preparation of Compound A is expected. The stable crystalline phosphate salt Compound (Compound A-P-Xln) preparation should contain the process of neutralization of Compound A by the solution of phosphoric acid in some type of solvent (or a mixture of solvents) and the process of recrystallization in some type of solvent (or a mixture of solvents). Preferably, this stable crystalline phosphate salt form of (Compound A-P-Xln) should be able to be accessed from the recrystallization of a solvent (or a mixture of solvents) which included but not limited to MeOH, EtOH and IPA.

### Example 8: Preparation of Enantiomers (Chiral Isomers)

Compound A (10 mg) was dissolved into IPA (10 ml) and the 2 ml solution was injected into a chiral HPLC machine with a chiral prep column CHIRALCEL OD 500×50 mm at flow rate 5 ml/min with the mobile phase Hexane:IPA:Diethyl Amine (85:15:0.1) at UV 240 nm. A total of five injections were repeated, and the collected fractions were combined, further evaporated to give Compound R-A (optical rotation: −23.49, 1.3 mg) and Compound S-A (optical rotation: +22.51, 1.6 mg) as two enantiomers.

R-A: ¹H-NMR (DMSO-d₆) δ ppm: 0.34-0.40 (m, 1H), 0.48-0.60 (m, 2H), 0.80-0.87 (m, 1H), 2.42 (s, 3H), 2.59-2.62 (d, 1H), 2.73-2.75 (d, 1H), 2.87-2.92 (m, 1H), 2.89 (t, 2H), 3.14-3.19 (m, 1H), 3.74-3.79 (m, 1H), 3.96 (s, 3H), 4.24 (t, 2H), 6.28 (s, 1H), 6.32-6.33 (d, 1H), 6.99 (t, 1H), 7.20-7.23 (d, 1H), 7.41 (s, 1H), 7.59 (s, 1H), 8.41-8.43 (d, 2H), 11.42 (s, 1H).

S-A: ¹H-NMR (DMSO-d₆) δ ppm: 0.34-0.41 (m, 1H), 0.49-0.59 (m, 2H), 0.80-0.85 (m, 1H), 2.42 (s, 3H), 2.59-2.62 (d, 1H), 2.73-2.76 (d, 1H), 2.87-2.92 (m, 1H), 2.89 (t, 2H), 3.14-3.19 (m, 1H), 3.74-3.79 (m, 1H), 3.96 (s, 3H), 4.24 (t, 2H), 6.28 (s, 1H), 6.32-6.33 (d, 1H), 6.99 (t, 1H), 7.20-7.23 (d, 1H), 7.41 (s, 1H), 7.59 (s, 1H), 8.41-8.43 (d, 2H), 11.42 (s, 1H).

### Example 9: Kinase Inhibition

To test the biological activity of 5-(2-(4-(4-fluoro-2-methyl-1H-indol-5-yloxy)-6-methoxyquinolin-7-yloxy)ethyl)-5-azaspiro[2.4]-heptan-7-ol (Compound A-Xln), the inhibitions of kinase FGFR1(h), FGFR2(h), FGFR3(h), Flt1(h) (VEGFr1), Flt4(h) (VEGFr3), KDR(h) (VEGFr2), Aurora-B PDGFRα(h), PDGFRα(h), PDGFRβ(h) were tested and the IC50 values were listed in the table below. Furthermore, the kinase inhibitions of a market drug Sunitinib were also tested and the results were compared with Compound A-Xln.

TABLE 9
Compound A kinase inhibitions comparing to Sunitinib

IC50 (nM)

| | | Compound |
|---|---|---|
| Kinase | Sunitinib | A-Xln |
| FGFR1(h) | 437 | 4 |
| FGFR2(h) | 852 | 8 |
| FGFR3(h) | 314 | 6 |
| Flt1(h) (VEGFr1) | 7 | 4 |
| Flt4(h) (VEGFr3) | 3 | 2 |
| KDR(h) (VEGFr2) | 34 | 15 |
| Aurora-B | — | 47 |
| PDGFRα(h) | | 160 |
| PDGFRβ(h) | | 26 |

— or blank: no inhibition or compound activity that could not be fit to an IC50 curve.

### Example 10

Ion chromatography analysis and water solubility results for the phosphoric salt of 5-(2-(4-(4-fluoro-2-methyl-1H-indol-5-yloxy)-6-methoxyquinolin-7-yloxy)ethyl)-5-azaspiro[2.4]-heptan-7-ol (Compound A-P) was listed in the table below.

TABLE 10
Ion chromatography analysis and water
solubility results for Compound A-P

| | | Phosphoric | Phosphoric |
|---|---|---|---|
| | Water | acid (Ion) | acid (Ion) |
| | Solubility | percentage | percentage |
| Name | (mg/ml) | (%) | (%) theory |
| Compound A-P | 0.36 | 17.35 | 17.03 |

### Example 11

In vitro MTT (proliferation) assay was performed with compound from above examples to give following inhibition results:

TABLE 11
Compound A-Xln inhibition results

| | | IC₅₀ (μM) |
|---|---|---|
| | Cell Line | Compound A-Xln |
| Ovary | SKOV3 | 17.1 ± 0.55 |
| | A2780 | 2.63 ± 0.27 |
| Blood | HL-60 | 1.98 ± 1.13 |
| | Molt-4 | 5.53 ± 2.78 |
| | K562 | 6.37 ± 2.56 |
| | U937 | 10.0 ± 2.28 |
| Breast | SKBR3 | 12.2 ± 2.00 |
| | MDA-MB-231 | 7.77 ± 0.02 |
| Lung | 95-D | 4.92 ± 2.26 |
| Liver | BEL-7402 | 0.86 ± 0.15 |
| | HepG2 | 9.12 ± 3.06 |
| | SMMC-7721 | 8.01 ± 2.53 |
| Kidney | 786-O | 10.1 ± 3.97 |
| Colon | HCT-116 | 11.7 ± 3.69 |
| Cervix | Hela | 11.2 ± 2.71 |
| Stomach | SGC7901 | 6.64 ± 0.81 |

TABLE 12
Compound A and Compound A-P in vitro inhibition activities against tumor cell lines

| | PANC- | NCI- | MDA- | | | |
|---|---|---|---|---|---|---|
| | 1 | H157 | MB-231 | Hela | PC-3 | BEL7404 |
| | IC50 | IC50 | IC50 | IC50 | IC50 | IC50 |
| | (μM) | (μM) | (μM) | (μM) | (μM) | (μM) |
| Cell Lines | pancreas | lung | breast | ovary | prostate | ovary |
| Compound A-P- | 2.75 | 1.61 | 3.93 | 2.36 | 1.77 | 1.75 |
| Xln | | | | | | |
| Compound R-A | 5.64 | 8.11 | 9.60 | 6.69 | 2.75 | 4.06 |
| Compound S-A | 5.0 | 7.0 | 6.60 | 6.30 | 5.60 | 2.38 |
| | MKN45 | Ishikawa | Saos-2 | SKOV3 | SW579 | HCT116 |
| | IC50 | IC50 | IC50 | IC50 | IC50 | IC50 |
| | (μM) | (μM) | (μM) | (μM) | (μM) | (μM) |
| Cell Lines | breast | endometrial | sarcoma | ovary | thyroid | colon |
| Compound A-P- | 0.13 | 1.48 | 2.48 | 3.09 | 0.880 | 0.934 |
| Xln | | | | | | |
| Compound R-A | 0.43 | 4.61 | 3.08 | 3.71 | 0.906 | 0.883 |
| Compound S-A | 0.32 | 4.98 | 5.35 | 7.65 | 0.836 | 0.980 |

### Example 12: Compound A-Xln in Vitro, in Vivo Combination Efficacies With Chemotherapy or Immunotherapy Against Tumor Cell Lines as Well as in Vivo Xenograft Model Against Various Tumor Cell Lines

Compound A-Xln combination therapy MTT assay results were investigated. 100 μl of cell suspension with a concentration of 5×104 cells/ml was added to each well of a 96-well plate, and place in a 37° C., 5% CO₂ incubator. After 24 h, Compound A-Xln sample solution was added according to Table 13, 10 μl/well for double-duplicate wells. After 72 hours at 37° C. and 5% CO₂ incubation, 20 μl of 5 mg/ml MTT solution was added to each well, then 100 μ1/well of dissolving solution was added after 4 hours. The samples were put in an incubator, and a full-wavelength multifunctional enzyme label machine was used after dissolving. The OD value at 570 nm was measured. FIGS. 13 and 14 shows the proliferation inhibition of breast cancer cell lines MCF7 and BT474, respectively, by Compound A-Xln, anti-breast cancer agent Anatrozole and Letrozole, combination of Compound A-Xln and Anatrozole, and combination of Compound A-Xln and Letrozole.

TABLE 13
Dosing list with two breast cancer cell lines MCF-7 and BT474

| | | Experimental | Dosing |
|---|---|---|---|
| Group | Compounds | time (h) | time |
| 1 | Compound A-Xln | 72 | Day 1 |
| | Letrozole | 72 | Day 1 |
| | Anastrozole | 72 | Day 1 |
| 2 | Compound A-Xln + | 72 | Day 1 |
| | Letrozole | | Day 1 |
| 3 | Compound A-Xln + | 72 | Day 1 |
| | Anastrozole | | Day 1 |

Samples according to Table 14 were also tested for proliferation inhibitor of three colon cancer lines HT29, HCT116 and colo205 by Compound A-Xln, anti-colon cancer agent 5FU/O, and combination of Compound A-Xln and 5FU/O. The results are shown in FIGS. 15-17.

TABLE 14
Dosing list with three colon cancer
cell lines HT29, HCT116 and colo205

| | | Experimental | Dosing |
|---|---|---|---|
| Group | Compounds | time (h) | time |
| 1 | Compound A-Xln | 72 | Day 1 |
| | 5-FU/Oxaliplatin | 72 | Day 1 |
| 2 | Compound A-Xln + | 72 | Day 1 |
| | 5-FU/Oxaliplatin | | Day 1 |
| 3 | Compound A-Xln + | 72 | Day 1 |
| | 5-FU/Oxaliplatin | (Compound A) | (Compound A) |
| | | 48 | Day 2 |
| | | (5-FU/Oxaliplatin) | (5-FU/Oxaliplatin) |
| 4 | 5-FU/Oxaliplatin + | 72 | Day 1 |
| | Compound A-Xln | (5-FU/Oxaliplatin) | (5-FU/Oxaliplatin) |
| | | 48 | Day 2 |
| | | (Compound A) | (Compound A) |

The efficacy of Compound A-Xln plus immunotherapies, such as anti-mouse PD-1 antibody combination treatment was also tested.

Nude mice were inoculated with murine colorectal CT26 cell line for 7 days and randomized into groups of 6 and were then treated with vehicle, Compound A-Xln in two dosing groups once daily, anti-mouse PD-1 (PD-1) at 200 μg/mouse once every 3 days in one group, or a combination of Compound A-Xln with anti-PD-1 in two dosing groups. Table 15 shows the change in the tumor size after administering the respective compounds. FIG. 18 shows a comparison of mean tumor volume changes following administration of Compound A-Xln, and anti-PD1 antibody agent, and combinational therapy of Compound A-Xln and an anti-PD1 antibody agent.

TABLE 15
Dosing list with murine colorectal CT26 cell

| | | Mean tumor | | Mean tumor | | |
|---|---|---|---|---|---|---|
| | Administration | volume (mm³) | | volume (mm³) | | T/C |
| Group | pathway | D0 | SEM | D17 | SEM | D17 |
| Vehicle | i.g. | 128.5 | 9.3 | 2281.8 | 219.3 | — |
| Compound A-Xln | i.g. | 127.8 | 14.1 | 1625.3 | 224.8 | 72.04% |
| 5 mg/kg/Qd | | | | | | |
| Compound A-Xln | i.g. | 128.5 | 15.1 | 829.0 | 168.3 | 36.67% |
| 15 mg/kg/Qd | | | | | | |
| PD-1 | IP | 126.5 | 11.5 | 1526.8 | 188.5 | 67.90% |
| 200 μg/mouse/Q3d | | | | | | |
| Compound A-Xln | i.g./IP | 128.3 | 15.0 | 1281.7 | 116.3 | 56.28% |
| 5 mg/kg/Qd + PD- | | | | | | |
| 1/200 μg/mouse/Q3d | | | | | | |
| Compound A-Xln | i.g./IP | 129.0 | 8.9 | 521.0 | 97.2 | 22.60% |
| 15 mg/kg/Qd + PD- | | | | | | |
| 1/200 μg/mouse/Q3d | | | | | | |

D0: the first drug administration time.
IP: intraperitoneal injection.
i.g.: Gastrointestinal administration.
Qd: once daily.
Q3d: once three days.
PD-1 (anti-PD-1): anti mouse PD-1 antibody

Compound A-Xln in vivo anti-tumor efficacies against various tumor cell lines on xenograft models have been tested.

Animal antitumor activity in vivo xenograft models with various tumor cell lines (SCLC NCI-H1436, NSCLC 95-D, ovarian SKVO3, renal 786-O, liver Bel-7420, brain U87) were performed as follows: The well grown tumor tissues of tumor cell lines were cut into 3 mm pieces, and each nude mouse was subcutaneously inoculated with one piece into the right flank. The animals were grouped and administered at pre-designed dosing. Treatments were initiated when the tumor size reached above 100 mm³ after 10-14 days. According to the size of tumor, the animals with oversize or undersize tumors were eliminated, and the animals were grouped with similar average tumor volume. Then the animals were orally administrated daily for continuous 14-21 days. The large diameter a (mm) and the small diameter b (mm) were measured with caliper twice a week after inoculation for 13 days. Each tumor volume was calculated by formular: TV=ab²/2 and the relative tumor volume was calculated as: RTV=Vt/Vo, Vo represents the tumor volume on the first day of treatment; Vt represents the tumor volume on each measurement day. The animals were executed and tumors were extracted by dissection 20-30 days after inoculation. The individual body weight and tumor weight were determined and calculated. Tumor Inhibit percentage was calculated as [1−((TWt)/(TWc)×100%, where TWt represents mean tumor weight of a treated group on the last day during the experiment, TWc represents mean tumor weight of the control group on the last day during the experiment. Experimental dosing and efficacy results are shown in FIGS. 19-24 of Drawing. The best tumor inhibition percentage ranges are >70% to 100%.

It is concluded that: Proliferation inhibition of human cancer cells, such as breast cancer cell lines MCF7 and BT474 by Compound A-Xln combined with anti-breast cancer agents, such as aromatase inhibitors Anatrozole and Letrozole, are demonstrated to be increased.

Proliferation inhibition of human cancer cells, such as colon cancer cell lines HT29, HCT116 and colo205 by Compound A-Xln combined with chemotherapy agents, such as anti-colon cancer 5FU/O, are demonstrated to be increased. The inhibition activity is also shown some difference in sequential dosing with higher activity on regimen of dosing chemotherapy agents first followed by dosing Compound A later.

Murine xenograft model demonstrates combined inhibition effect against cancer cell proliferation, such as murine colorectal cancer cell line CT26 proliferation. Combination treatment of Compound A-Xln with an anti-PD1 antibody agent against cancer cell proliferation provides strong evidence of increased efficacy in an in vivo animal model.

Combination therapy of Compound A-Xln with immunotherapeutic agents such as PD-1 or PD-L1 antibody, such as those selected from, but not limited to, nivolumab, pembrolizumab, ipilimumab, blinatumomab, elotuzumab, daratumumab, cemiplimab, avelumab, durvalumab, atezolizumab, toripalimab, sintilimab, camrelizumab, tislelizumab, AK104, AK105, Penpulimab, KN035, CS1001, talimogene laherparepvec, can generate synergistic clinical efficacy.

Various human cancer cell line animal xenograft models have demonstrated remarkable tumor inhibition activities from Compound A-Xln, especially in SCLC, NSCLC, ovarian, renal, liver and brain cancers.

### Example 13: Compound A-Xln tablet in Human Clinical Trials Against Solid Tumors, Such as NSCLC, SCLC, Ovarian, Endometrial, Cervical, Uterine Leiomyosarcoma and Peritoneal Mesothelioma Cancers

### A: Overview

Compound A-Xln tablet was administrated orally at 90 mg or 80 mg or 70 mg or 60 mg or 40 mg once a day for 28 days as one cycle for various cancer patients until intolerability or progression disease (PD) via RECISIT 1.1 evaluation. 90 mg daily can be reduced in 10 mg interval to be 80 mg or 70 mg or 60 or 50 mg or 40 mg or 30 mg or 20 mg daily in case of intolerability observed. Treatment results with few representative patients with remarkable unexpected antitumor activities listed below (Cutoff, March 2022). Very positive Objective Response Rate (ORR) for SCLC for ≥3rd line (≥2nd prior lines) treatment is observed at ≥20% and for Platinum resistant ovarian cancer for ≥3rd line (≥2nd prior lines) treatment is observed around 20%. There are some efficacies for SCLC patients who were on maintenance therapy with Compound A-Xln tablet after 1st line standard of care (SOC) therapy cycles finish. There are some efficacies for SCLC patients who have been treated with Compound A-Xln tablet as 2nd line (prior 1 line) treatment option. Efficacies were observed in sarcoma and brain tumors as well.

| | | PFS | Best Response (-% | Prior |
|---|---|---|---|---|
| Subject # | Indication | (months) | tumor reduction) | Lines |
| 20019 | NSCLC | 25 | SD (−20%) | >3 |
| S01006-A | SCLC | *11 | SD (−4.5%) | 1 (Mnt) |
| S02008 | SCLC | *5.6 | SD (−28.96%) | 1 (Mnt) |
| S08004-B | SCLC | *15 | PR (−45.6%) | 1 |
| S08008-B | SCLC | 7.4 | PR (−55.7%) | 1 |
| S08011-B | SCLC | 9.4 | PR (−53.6%) | 1 |
| S08012-B | SCLC | 9.3 | PR (−46.6%) | 1 |
| S05001-C | SCLC | *13 | PR (−43.5%) | 2 |
| S08002-C | SCLC | 5.6 | PR (−44.8%) | 2 |
| S08001-C | SCLC | 4.77 | PR (−32.1%) | 2 |
| S08006-C | SCLC | 3.73 | PR (−40.2%) | 2 |
| S08021-C | SCLC | *2.1 | PR (−40.7%) | 2 |
| S01013 | OC | *1.9 | PR (−46.9%) | 2 |
| S01003 | OC | 7.37 | PR (−83.5%) | 3 |
| S0302008 | ULMS | 13.1 | SD (0%) | 3 |
| S0302015 | BT | 6 | SD (−15%) | 1 |

*non progression disease time/still in study.
Mnt: maintenance therapy

Furthermore, a completed SCLC clinical study with thirty patients with prior 1st line (Group B) and 30 patients with ≥2nd prior line (Group C) has demonstrated positive clinical efficacies on Progression Free Survival (PFS) of B: 3.68 months and C: 3.62 months, and Overall Survival (OS) of B: 10.39 months and C: >15 months. They are the much better than current historic reported available treatment options for the prior 1st line or ≥2nd line treatments. Kaplan-Meier curves have been shown in the Drawing as FIG. 25 and FIG. 26.

### B: Representative Subject Examples (10 mg Tablet Each was Used)

### (1): Representative Subject S01013 in Phase 1b

Subject S01013 with NSCLC (prior line>3) was orally administered with Compound A-Xln tablet 60 mg (10 mg×6) once daily in 28-day cycles. The dose was reduced to 40 mg and 30 mg at the second cycle, and similar aforementioned treatment regimen was used in the following cycles. The subject's best response of the treatment was PR (by 54.70% reduction compared to baseline target lesions).

### (2): Representative Subject S01014 in Phase 1b

Subject S01014 with NSCLC (prior line=3) was orally administered with Compound A-Xln tablet 60 mg once daily in 28-day cycles. The dose was reduced to 40 mg at the third cycle, and a similar aforementioned treatment regimen was used in the following cycles. The subject best response of the treatment was PR (by 56.95% reduction compared to baseline target lesions).

### (3): Representative Subject S05002 in Phase 1b

Subject S05002 with NSCLC (prior line=2) was orally administered with Compound A-Xln tablet 60 mg once daily in 28-day cycles. A similar aforementioned treatment regimen was used in the following cycles. The subject's best response of the treatment was PR (by 38.42% reduction compared to baseline target lesions).

### (4): Representative Subject 020207 in Phase 1

Subject 020207 with peritoneal mesothelioma (prior line=3) was orally administered with Compound A-Xln tablet 40 mg once daily in 28-day cycles in combination with Toripalimab Injection (240 mg, once in 3 weeks). A similar aforementioned treatment regimen was used in the following cycles. The subject's best response of the treatment was PR by 36.4% reduction compared to baseline target lesions).

### (5): Representative Subject 020202 in Phase 1

Subject 020202 with small-cell lung carcinoma (prior line=1) was orally administered with Compound A-Xln tablet 40 mg once daily in 28-day cycles in combination with Toripalimab Injection (240 mg, once in 3 weeks). A similar aforementioned treatment regimen was used in the following cycles. The subject's best response of the treatment was PR (by 38.22% reduction compared to baseline target lesions).

What is claimed is:

1. A stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) is made by a method comprising subjecting an amorphous form of 5-(2-(4-(4-fluoro-2-methyl-1H-indol-5-yloxy)-6-methoxyquinolin-7-yloxy)ethyl)-5-azaspiro[2.4]-heptan-7-ol to recrystallization, selected but not limited to using a high boiling point solvent or a mixture of solvents that together have a high boiling point, and using a low boiling point solvent or a mixture of solvents that together have a low boiling point, wherein the high boiling point solvent is DMF and the low boiling point solvent is EtOH.

2. A stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) according to claims 1 exhibiting at least one of the following properties:
a DSC Melting Range (Endo): 240-260° C. with Peak Temp Range: 244-254° C.;
and a specifical DSC Melting Range (Endo): 247-253° C. with Peak Temp of 249° C.; and.
a TGA thermogram demonstrating as an unsolvated material with weight loss above 250° C.

3. A stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) according to claims 1 featuring a XRPD pattern compromising 14 characteristic peaks with intensity % greater than 10% at 8.9, 9.9, 14.9, 15.4, 16.1, 16.6, 18.5, 19.9, 20.4, 21.5, 23.9, 24.3, 24.6, and 26.0 degrees two-theta.

4. A stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) according to claims 1 featuring a XRPD pattern compromising 43 characteristic peaks with all intensity % expressed in d values and angles as follows:

| NO. | Angle | d value | Intensity | Intensity % |
|---|---|---|---|---|
| 1 | 8.899 | 9.9286 | 74540 | 12.04 |
| 2 | 9.927 | 8.9032 | 235708 | 38.08 |
| 3 | 11.586 | 7.6312 | 14908 | 2.41 |
| 4 | 13.145 | 6.7299 | 3511 | 0.57 |
| 5 | 14.938 | 5.9257 | 159717 | 25.80 |
| 6 | 15.374 | 5.7587 | 104484 | 16.88 |
| 7 | 16.104 | 5.4991 | 93019 | 15.03 |
| 8 | 16.558 | 5.3495 | 619004 | 100.00 |
| 9 | 17.327 | 5.1137 | 22260 | 3.60 |
| 10 | 17.820 | 4.9734 | 20322 | 3.28 |
| 11 | 18.492 | 4.7942 | 87559 | 14.15 |
| 12 | 18.809 | 4.7140 | 21872 | 3.53 |
| 13 | 19.912 | 4.4553 | 118882 | 19.21 |
| 14 | 20.445 | 4.3403 | 180671 | 29.19 |
| 15 | 21.511 | 4.1275 | 348507 | 56.30 |
| 16 | 22.004 | 4.0362 | 14250 | 2.30 |
| 17 | 22.499 | 3.9485 | 3195 | 0.52 |
| 18 | 23.188 | 3.8327 | 51679 | 8.35 |
| 19 | 23.859 | 3.7265 | 69270 | 11.19 |
| 20 | 24.314 | 3.6577 | 67756 | 10.95 |
| 21 | 24.590 | 3.6172 | 93287 | 15.07 |
| 22 | 25.171 | 3.5350 | 1707 | 0.28 |
| 23 | 25.952 | 3.4305 | 86263 | 13.94 |
| 24 | 26.464 | 3.3652 | 53165 | 8.59 |
| 25 | 26.841 | 3.3188 | 20232 | 3.27 |
| 26 | 27.217 | 3.2738 | 6582 | 1.06 |
| 27 | 27.648 | 3.2237 | 46760 | 7.55 |
| 28 | 28.457 | 3.1339 | 54339 | 8.78 |
| 29 | 29.310 | 3.0446 | 1984 | 0.32 |
| 30 | 29.856 | 2.9901 | 4264 | 0.69 |
| 31 | 30.212 | 2.9557 | 18700 | 3.02 |
| 32 | 31.202 | 2.8642 | 26858 | 4.34 |
| 33 | 31.632 | 2.8262 | 24054 | 3.89 |
| 34 | 32.837 | 2.7252 | 55773 | 9.01 |
| 35 | 33.430 | 2.6782 | 39092 | 6.32 |
| 36 | 35.185 | 2.5485 | 6426 | 1.04 |
| 37 | 36.092 | 2.4865 | 16774 | 2.71 |
| 38 | 36.865 | 2.4361 | 20105 | 3.25 |
| 39 | 38.167 | 2.3560 | 16104 | 2.60 |
| 40 | 39.409 | 2.2845 | 5045 | 0.82 |
| 41 | 40.888 | 2.2053 | 5238 | 0.85 |
| 42 | 41.720 | 2.1632 | 8720 | 1.41 |
| 43 | 42.886 | 2.1070 | 9964 | 1.61 |

5. A stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) according to claims 1 featuring a XRPD pattern substantially as depicted in FIG. 4.

6. A stable crystalline form (Compound A-P-Xln) of a phosphoric acid salt of crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) according to claims 1, wherein the phosphoric acid salt is prepared by a method including a step for neutralization of a solution of phosphoric acid in a solvent or a mixture of solvents and the process of recrystallization.

7. The compound of claim 6, wherein the solvent in which neutralization and/or recrystallization is performed in EtOH.

8. The compound of claims 6, wherein the phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol is characterized by one or more of the following properties:
a DSC Melting Range (Endo): 221-235° C. with a Peak Temp of 229° C.; and
a TGA demonstrating as a slight weight loss at about 30-60° C. and significantly weight loss above 210° C.

9. The compound of claims 6, wherein the phosphoric acid salt of 5-(2-(4-(4-fluoro-2-methyl-1H-indol-5-yloxy)-6-methoxyquinolin-7-yloxy)ethyl)-5-azaspiro[2.4]-heptan-7-ol featuring a XRPD pattern compromising all characteristic peaks with all intensity % expressed in d values and angles as follows:

| NO. | Angle | d value | Intensity | Intensity % |
|---|---|---|---|---|
| 1 | 5.268 | 16.760 | 83.1 | 26.6 |
| 2 | 7.139 | 12.372 | 251.0 | 80.6 |
| 3 | 9.805 | 9.014 | 94.9 | 30.4 |
| 4 | 10.455 | 8.455 | 119.0 | 38.2 |
| 5 | 11.799 | 7.494 | 98.1 | 31.4 |
| 6 | 12.417 | 7.123 | 181.0 | 58.1 |
| 7 | 12.669 | 6.982 | 225.0 | 72.0 |
| 8 | 13.672 | 6.472 | 280.0 | 89.8 |
| 9 | 14.307 | 6.186 | 136.0 | 43.5 |
| 10 | 15.546 | 5.695 | 137.0 | 44.0 |
| 11 | 16.064 | 5.513 | 181.0 | 58.1 |
| 12 | 16.719 | 5.298 | 312.0 | 100.0 |
| 13 | 17.495 | 5.065 | 135.0 | 43.4 |
| 14 | 18.035 | 4.915 | 136.0 | 43.7 |
| 15 | 18.802 | 4.716 | 153.0 | 48.9 |
| 16 | 19.530 | 4.542 | 285.0 | 91.3 |
| 17 | 21.095 | 4.208 | 200.0 | 64.1 |
| 18 | 22.535 | 3.942 | 138.0 | 44.2 |
| 19 | 23.426 | 3.794 | 294.0 | 94.3 |
| 20 | 25.916 | 3.435 | 114.0 | 36.6 |
| 21 | 26.577 | 3.351 | 144.0 | 46.3 |

10. A compound having a structure represented by 5-(2-(4-(4-fluoro-2-methyl-1H-indol-5-yloxy)-6-methoxyquinolin-7-yloxy)ethyl)-5-azaspiro[2.4]-heptan-7-ol, its stable crystalline form, its salt form, and/or its stable crystalline salt form, and/or its enantiomers according to claims 1, for use in inhibiting protein tyrosine kinases (PTKs).

11. The compound of claim 10, wherein the PTKs are selected but not limited to FGFR1(h), FGFR2(h), FGFR3(h), Flt1(h) (VEGFr1), Flt4(h) (VEGFr3), KDR(h) (VEGFr2), Aurora-B PDGFRα(h), PDGFRα(h) and PDGFRβ(h).

12. A compound having a structure represented by 5-(2-(4-(4-fluoro-2-methyl-1H-indol-5-yloxy)-6-methoxyquinolin-7-yloxy)ethyl)-5-azaspiro[2.4]-heptan-7-ol, its stable crystalline form, its salt form, and/or its stable crystalline salt form, and/or its enantiomers according to claims 1 wherein the compound inhibits common cancer cell lines of pancreas, prostate, sarcoma, thyroid, colon, ovary, blood, breast, brain, NSCLC, SCLC, liver, kidney, colon, cervix and stomach selected but not limited to the group consisting of PANC-1, NCI-H157, MDA-MB-231, Hela, PC-3, BEL7404, MKN45, Ishikawa, Saos-2, SKOV3, SW579, NCI-H1436, Bel-7402, U87 and HCT116.

13. A compound according to claim 1 having a structure represented selected by S-5-(2-(4-(4-fluoro-2-methyl-1H-indol-5-yloxy)-6-methoxyquinolin-7-yloxy)ethyl)-5-azaspiro[2.4]-heptan-7-ol and R-(2-(4-(4-fluoro-2-methyl-1H-indol-5-yloxy)-6-methoxyquinolin-7-yloxy)ethyl)-5-azaspiro[2.4]-heptan-7-ol and their phosphoric acid salt form.

14. A pharmaceutical composition that comprises an active ingredient that is a compound of any one of claims 1, 6 and 13 and a pharmaceutically acceptable carrier and/or pharmaceutically acceptable excipient.

15. A pharmaceutical composition contains a compound of claim 1 that comprises 10 mg or 30 mg stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) and a pharmaceutically acceptable carrier selected but not limited to a group of Lactose T80, Microcrystalline cellulose (PH101). Sodium Carboxymethyl Starch, Silicon dioxide, Magnesium Stearate, Opadry gastric-soluble coating powder to form a tablet.

16. A method of treating a neoplastic disease, comprising administering a stable crystalline form (Compound A-Xln) of 5-(2-(4-(4-fluoro-2-methyl-1H-indol-5-yloxy)-6-methoxyquinolin-7-yloxy)ethyl)-5-azaspiro[2.4]-heptan-7-ol (Compound A) or pharmaceutical composition comprising a stable crystalline form (Compound A-Xln) and pharmaceutically acceptable excipients to a subject in need thereof.

17. A method of treating as claimed in claim 16, wherein the treatment further comprises administration of an additional agent, the additional agent being a chemotherapy compound and/or immunotherapy agents.

18. A method of treating as claimed in claim 17, wherein the combination chemotherapy agents are selected but not limited to a group of platinum-based or taxane-based or topoisomerase 1 inhibitor or alkaloid or alkylating agents consisting of cisplatin, carboplatin, paclitaxel or cisplatin/paclitaxel or carboplatin/paclitaxel or carboplatin/etoposide or topotecan, irinotecan or lomustine.

19. A method of treating as claimed in claim 17, wherein the combing immunotherapy agents are selected but not limited to a group of PD-1 or PD-L1 antibodies consisting of nivolumab, pembrolizumab, ipilimumab, blinatumomab, elotuzumab, daratumumab, cemiplimab, avelumab, durvalumab, atezolizumab, toripalimab, sintilimab, camrelizumab, tislelizumab, AK104, AK105, penpulimab, KN035, CS1001, talimogene laherparepvec.

20. A method of treating as claimed in claim 16, wherein the neoplastic disease is solid tumors, selected but not limited to a group of NSCLC, SCLC, mesothelioma, renal, colorectal, gastric, melanoma, head/neck, thyroid, pancreatic, liver, prostate, bladder, brain, sarcoma, breast, ovarian, cervical and endometrial cancers; and blood cancers, selected but not limited to a group of ALL, CLL, AML, CML and Multiple Myeloma.

21. A method of treating as claimed in claim 16, wherein the neoplastic disease is small cell lung cancer, non-small cell lung cancer, mesothelioma, ovarian cancer, brain tumor, and sarcoma.

22. A method of treating as claimed in claim 16, where the treatment is using Compound A-Xln tablet for 1st line patient maintenance therapy or for 2nd or ≥3rd line patient treatment.

23. A method of treating as claimed in claims 16 and 17, wherein the treatment dosing is daily at mg or 80 mg or 70 mg or 60 mg or 50 mg or 40 mg or 30 mg or 20 mg.
